(19) United States
(12) Patent Application Publication
(10) Pub. No.: US 2019/0161421 A1
(43) Pub. Date: May 30, 2019

(54) Hydrocarbon Pyrolysis

(71) Applicant: ExxonMobil Chemical Patents Inc., Baytown, TX (US)

(72) Inventors: Federico Barrai, Houston, TX (US); John S. Coleman, Houston, TX (US); Dhaval A. Bhandari, Bridgewater, NJ (US); Frank Hershkowitz, Basking Ridge, NJ (US); James R. Lattner, La Porte, TX (US)

(21) Appl. No.: 16/320,655

(22) PCT Filed: Aug. 15, 2017
(86) PCT No.: PCT/US2017/046937

§ 371 Date: Jan. 25, 2019

Related U.S. Application Data
(60) Provisional application No. 62/381,722, filed on Aug. 31, 2016.

(30) Foreign Application Priority Data
Oct. 17, 2016 (EP) 16194181.0

(51) Int. Cl. C07C 5/327; C01B 3/22; B01F 5/06

(52) U.S. Cl. CPC C07C 5/327; B01J 2208/00513; B01F 5/0688; C01B 3/22

(57) ABSTRACT

The invention relates to hydrocarbon pyrolysis, to equipment and materials useful for hydrocarbon pyrolysis, to processes for carrying out hydrocarbon pyrolysis, and to the use of hydrocarbon pyrolysis for. e.g., natural gas upgrading. The pyrolysis can be carried out in a reverse-flow reactor.

### CROSS-REFERENCE TO RELATED APPLICATIONS

This application claims the benefit of U.S. Patent Application Ser. No. 62/381,722, filed Aug. 31, 2016, and European Patent Application No. 16194181.0, filed Oct. 17, 2016, which are incorporated herein by reference. Cross reference is made to the following related patent applications: U.S. Patent Application Ser. No. 62/402,009, filed Sep. 30, 2016, U.S. Patent Application Ser. No. 62/466,050, filed Mar. 2, 2017, U.S. Patent Application Ser. No. 62/486,545, filed Apr. 18, 2017, PCT Patent Application No. PCT/US2017/046871, (Docket No. 2017EM233PCT), filed Aug. 15, 2017, and PCT Patent Application No. PCT/US2017/046879, (Docket No. 2017EM234PCT), filed Aug. 15, 2017, which are incorporated by reference herein.

### FIELD

The invention relates to hydrocarbon pyrolysis, to equipment and materials useful for hydrocarbon pyrolysis, to processes for carrying out hydrocarbon pyrolysis, and to the use of hydrocarbon pyrolysis for, e.g., natural gas upgrading. The pyrolysis can be carried out in a reverse-flow reactor.

### BACKGROUND

Olefinic compounds are a class of hydrocarbon compounds which have at least one double bond of four shared electrons between two carbon atoms. In part as a result of their utility as feeds for producing desirable products, olefin demand to grow, particularly for light olefin such as ethylene, propylene, and butenes.

Light olefin can be produced in commercial quantities by steam cracking of hydrocarbon-containing feeds. A steam cracker generally includes a plurality of tubular members (typically referred to as “furnace tubes”) located proximate to one or more fired heaters which heat the outer surface of the furnace tubes. A mixture of the feed and steam is introduced into the heated furnace tubes, and heat transferred from the furnace tube to the mixture results in the conversion of at least a portion of the feed to light olefin by pyrolysis. Conditions in the steam cracker are controlled, typically at a temperature in the range of 750° C. to 900° C., to achieve a fixed, predetermined feed conversion, typically in the range of about 60% to about 80%. Although ethylene is the primary light olefin product of steam cracking, the process can also produce appreciable yields of propylene and butenes, particularly when the feed comprises C₅₊ hydrocarbon. Since steam cracking process conditions are selected to provide a fixed, predetermined feed conversion, ethylene, propylene and butylene yields are substantially constant. Besides light olefin, steam cracking also produces appreciable yields of molecular hydrogen, methane, ethane, propane, butanes, acetylene, butadiene, and C₅₊ saturated and unsaturated hydrocarbon, including coke. Steam crackers typically include facilities for recovering light olefin from the steam cracker's effluent.

The amount of steam present in the feed-steam mixture is generally in the range of about 0.2 pounds of steam per pound of hydrocarbon feed (0.09 kg/kg) to 0.7 lbs. of steam per pound of hydrocarbon feed (0.32 kg/kg). Typically, steam pressure is in the range of about 30 lbs. per sq. in. (psig, 207 kPag) to about 80 psig (552 kPag). The presence of steam lessens the amount of coke produced during the pyrolysis by decreasing hydrocarbon partial pressure. Steam in the feed-steam mixture also reacts with coke and coke precursors during the pyrolysis, which further decreases the amount coke produced during the pyrolysis. Even with added steam, however, the pyrolysis produces an appreciable yield of coke and coke precursors, and a portion of the coke accumulates in the furnace tubes.

Accumulating coke leads to both an undesirable pressure-drop increase across the tubes' internal flow path and a decrease in heat transfer to the feed-steam mixture. To overcome these difficulties, at least a portion of accumulated coke is removed from the interior of a tube by switching the tube from pyrolysis mode to decoking mode. During decoking mode, the flow of feed-steam mixture into the tube is terminated, and a flow of decoking fluid is established instead. The decoking fluid, typically comprising air and/or steam, reacts with and removes the accumulated coke. When sufficient coke has been removed, the tube is switched from decoking mode to pyrolysis mode to resume light olefin production. Although periodic decoking mode operation is effective for lessening the amount of accumulated coke, this benefit is obtained at a substantial energy cost. In part to lessen damage to the furnace tubes, e.g., by repeated thermal expansion/contractions, the fired heaters operate not only during pyrolysis mode, but also during decoking mode, even though an appreciable amount of recoverable light olefin is not produced during decoking mode.

In order to increase energy efficiency and improve the yield of light unsaturated hydrocarbon, processes have been developed which carry out the pyrolysis in a regenerative pyrolysis reactor. Such reactors generally includes a regenerative thermal mass having at least one internal channel, similar to the channeled tubular members described in U.S. Pat. Nos. 9,346,728; 9,187,382; 7,943,808; 7,846,401; 7,815,873; 9,322,549; and in U.S. Patent Application Publications Nos. 2015-0197696 and 2014-0303416. The thermal mass is preheated, and then a flow of the hydrocarbon-containing feed is established through the channel. Heat is transferred from the thermal mass to the hydrocarbon feed, which increases the hydrocarbon feed's temperature and results in conversion of at least a portion of the feed by pyrolysis. The pyrolysis produces a pyrolysis product comprising molecular hydrogen, methane, acetylene, ethylene, and C₃₊ hydrocarbon, which includes coke and coke precursors. At least a portion of the coke remains in the passages of the thermal mass, and the remainder of the pyrolysis product is conducted away from the reactor as a pyrolysis effluent. Ethylene is typically recovered from the pyrolysis effluent downstream of the reactor. Since the pyrolysis is endothermic, pyrolysis mode operation will eventually cool the thermal mass, e.g., to a temperature at which the pyrolysis reactions terminate. The ability to carry out pyrolysis reactions is restored by regenerating the thermal mass during a heating mode. During heating mode, the flow of hydrocarbon-containing feed to the regenerative pyrolysis reactor is terminated. Flows of oxidant and fuel are established to the reactor, typically in a direction that is the reverse of the feed flow direction, and heat is transferred from combustion of the fuel and oxidant to thermal mass to reheat the thermal mass. After sufficient heat is transferred from the combustion to the thermal mass, which reheats the thermal mass, the reactor is switched from reheating mode to pyrolysis mode.

More recently, U.S. Patent Application Publication No. 2016-176781 discloses increasing ethylene yield from a regenerative pyrolysis reactor by operating the pyrolysis mode in a regenerative pyrolysis reactor at a temperature in the range of 850° C. to 1200° C. and a hydrocarbon partial pressure ≥7 psia (48 kPa). The reference (e.g., in its FIG. 1A) discloses controlling the pyrolysis mode for increased ethylene selectivity and decreased selectivity for coke and methane by establishing a sharp thermal gradient in the bulk gas temperature profile between a region of substantially constant temperature at which the pyrolysis can occur and a substantially constant lower temperature at which pyrolysis does not occur. Although utilizing such pyrolysis conditions results in a coke yield that is less than that of steam cracking, some coke does accumulate in the channel. Advantageously, the reference reports that accumulated coke can be oxidized to volatile products such as carbon dioxide during heating mode by combustion using a portion of the oxidant in the oxidant flow. Energy efficiency is increased over steam cracking because (i) heating is not needed during pyrolysis mode and (ii) heat released by coke combustion in passages of the thermal mass during heating mode aids thermal mass regeneration. Although the process is more energy efficient than steam cracking, maintaining a sharp temperature gradient in the bulk gas temperature profile leads to substantially constant ethylene and C₃₊ hydrocarbon selectivities being maintained over the length of the pyrolysis zone during pyrolysis mode.

Energy efficient processes are now desired which have flexibility to produce a range of product selectivities over the length of the pyrolysis zone during pyrolysis mode, particularly processes which exhibit appreciable feed conversion without excessive coke yield.

### SUMMARY OF THE INVENTION

The invention is based in part on the discovery that regenerative pyrolysis reactors can be operated to produce an appreciable range of product selectivities, particularly light unsaturated hydrocarbon selectivities, over the length of the pyrolysis zone during pyrolysis mode, with appreciable feed conversion and without excessive coke yield. Unexpectedly, and contrary to the teachings of the prior art, a bulk gas temperature profile containing a sharp thermal gradient is not needed for effectively controlling the pyrolysis. Instead, it has been found that a bulk gas temperature profile which continuously varies over the length of a thermal mass in the pyrolysis zone does not lead to an appreciable loss of process control that would otherwise diminish conversion and produce excessive yields of less-desired or undesired products. A continuously varying bulk gas temperature profile within the pyrolysis zone during the pyrolysis results in a range of light olefin selectivities, which provides operational flexibility to produce yield variations for desirable light olefin compounds. More particularly, it has been found that it is beneficial for the gaseous hydrocarbon undergoing pyrolysis, e.g., within or proximate to thermal mass, within the pyrolysis zone to not have a sharp gradient in its temperature profile. Moreover, it has been unexpectedly found that it can be undesirable for the bulk gas temperature during profile pyrolysis mode to exhibit a substantially-constant high temperature in a first region of the pyrolysis zone and a substantially constant lower temperature in a second region of the pyrolysis zone, particularly where the bulk gas temperature profile exhibits an abrupt temperature gradient between the first and second regions.

Accordingly, certain aspects of the invention relate to a hydrocarbon pyrolysis process carried out in at least one elongated flow-through reactor of length L_{R}. The reactor has an internal volume which includes at least two regions. The reactor also includes at least one heated thermal mass located in the first region, wherein the thermal mass has a length L_{M}, and L_{M} is at least 0.1*L_{R}. The thermal mass has at least one internal channel, and first and second apertures in fluidic communication with the channel. The first and second apertures are separated by a flow-path of length L_{M} through the channel. The first aperture is proximate to the first opening. A flow of the feed is established via the first opening from the first aperture into the channel and toward the second aperture. The feed comprises ≥1 wt. % of C₂₊ hydrocarbon. The feed flow's C₂₊ hydrocarbon is pyrolysed in the channel under pyrolysis conditions to produce a flow of a pyrolysis product comprising molecular hydrogen, ethylene, propylene, and butadiene. The pyrolysis conditions include a bulk gas temperature profile which at the start of the pyrolysis increases substantially monotonically from a first temperature (T₁) proximate to the first aperture to a second temperature (T₂) proximate to the second aperture, T₂ being in the range of from 800° C. to 1400° C. The pyrolysis conditions also include a peak gas temperature Tₚ within the internal volume, wherein Tₚ is located in the second region. Tₚ is >T₂. The pyrolysis conditions result in a conversion profile for the feed's C₂₊ hydrocarbon, particularly for the saturated components thereof, which at the start of the pyrolysis increases from a first conversion (X₁) at a reference location R₁ positioned between the first and second apertures to a second conversion (X₂) proximate to the second aperture, wherein X₁ is in the range of from 25% to 85%, and X₂ is in the range of from 65% to 98%. The flow of the pyrolysis product is conducted out of the second aperture.

In other aspects the reactor and feed can be similar to those of the preceding aspects, but a different temperature profile is used. Pyrolysis mode is carried out for a time duration (“t_{P}”) that is substantially equal to t₂−t₁. At time t₁, a flow of feed is initiated into the heated thermal mass at the first aperture and toward the second aperture. At a later time t₂, the flow of feed is terminated. The pyrolysis conditions include a bulk gas temperature profile which at t₁ continuously varies (with no sharp gradients) from a first temperature (T₁) at the first aperture to a second temperature (T₂) at the second aperture. The pyrolysis conditions also include a peak gas temperature Tₚ in the internal volume. In these aspects, Tₚ occurs at a location along L_{M}, with Tₚ being >T₂, and T₂ being >T₁. T₂ is in the range of from 800° C. to 1400° C. The pyrolysis conditions result in a conversion profile for the feed's saturated C₂₊ hydrocarbon which at t₁ continuously varies from a minimum conversion (X₁) at a reference location between the first and second apertures to a second conversion (X₂) at the second aperture. At the location of Tₚ the conversion has a peak Xₚ>X₂. At t₁, X₂ is in a range of from 55% to 95% and Xₚ is ≤98%. During t_{P}, the conversion profile exhibits a continuously decreasing conversion at the second aperture and a continuously decreasing peak conversion. A flow of the pyrolysis product is conducted out of the second aperture and away from thermal mass during t_{P}.

In still other aspects, the invention relates to a regenerative pyrolysis reactor for carrying out any of the preceding aspects, and to the pyrolysis products produced by the pyrolysis of any of the preceding aspects.

### BRIEF DESCRIPTION OF THE FIGURES

FIG. 1 schematically shows one form of a reverse flow reactor that is suitable for carrying out certain aspects of the invention.

FIGS. 2-5 schematically show forms of a reverse flow reactor and representative bulk gas temperature profiles at the start (solid lines) and end (dashed lines) of pyrolysis.

FIG. 6 shows the variation of conversion as a function of Tₐᵥ for the reverse flow reactor of FIG. 2 during pyrolysis mode.

FIGS. 7-9 show the selectivities of the desired hydrocarbon compounds for the reverse flow reactor of FIG. 2 during pyrolysis mode.

### DETAILED DESCRIPTION

### Definitions

For the purpose of this description and appended claims, the following terms are defined.

The term “Cₙ” hydrocarbon means hydrocarbon having n carbon atom(s) per molecule, wherein n is a positive integer. The term “Cₙ₊” hydrocarbon means hydrocarbon having at least n carbon atom(s) per molecule. The term “Cₙ₋” hydrocarbon means hydrocarbon having no more than n carbon atom(s) per molecule. The term “hydrocarbon” means a class of compounds containing hydrogen bound to carbon, and encompasses (i) saturated hydrocarbon. (ii) unsaturated hydrocarbon, and (iii) mixtures of hydrocarbons, and including mixtures of hydrocarbon compounds (saturated and/or unsaturated), such as mixtures of hydrocarbon compounds having different values of n.

The terms “alkane” and “paraffinic hydrocarbon” mean substantially-saturated compounds containing hydrogen and carbon only, e.g., those containing ≤1% (molar basis) of unsaturated carbon atoms. The term “unsaturate” and “unsaturated hydrocarbon” refer to one or more C₂₊ hydrocarbon compounds which contain at least one carbon atom directly bound to another carbon atom by a double or triple bond. The term “olefin” refers to one or more unsaturated hydrocarbon compound containing at least one carbon atom directly bound to another carbon atom by a double bond. In other words, an olefin is a compound which contains at least one pair of carbon atoms, where the first and second carbon atoms of the pair are directly linked by a double bond. The term “aromatics” and “aromatic hydrocarbon” mean hydrocarbon compounds containing at least one aromatic ring.

The term “selectivity” refers to the production (weight basis) of a specified compound in a reaction. As an example, the phrase “a hydrocarbon pyrolysis reaction has 100% selectivity for methane” means that 100% of the hydrocarbon (weight basis) that is convened in the pyrolysis reaction is converted to methane. When used in connection with a specified reactant, the term “conversion” means the amount of the reactant (weight basis) consumed in the reaction. For example, when the specified reactant is ethane, 100% conversion means 100% of ethane is consumed in the reaction. With respect to hydrocarbon pyrolysis the term “conversion” encompasses any molecular decomposition by at least pyrolysis heat, including cracking, breaking apart, and reformation. Average conversion in a reaction zone, e.g., a pyrolysis zone, is the conversion achieved at Tₐᵥ. Yield (weight basis) is conversion times selectivity.

The term “pyrolysis” means an endothermic reaction for converting molecules into (i) atoms and/or (ii) molecules of lesser molecular weight, and optionally (iii) molecules of greater molecular weight, e.g., processes for converting ethane and/or propane to molecular hydrogen and unsaturates such as ethylene, propylene and acetylene. Certain aspects of the invention feature a pyrolysis zone exhibiting selectivities (e.g., of desired products) which vary as a function of position along the length of the pyrolysis zone but which do not vary appreciably as a function of time during pyrolysis mode, e.g., within about +/−25%, such as +/−10%, or +/−5% from selectivity at the start of t_{P}. More particularly, for certain aspects in which Tₐᵥ and/or Tₚ decrease by ≤100° C. during pyrolysis mode, the yield of many desired products, e.g., light olefin yield, such as ethylene and/or propylene yield, do not vary appreciably as a function of time during pyrolysis mode even though the product selectivities vary as a function of position along the length of the pyrolysis zone. For example, yield is typically within about +/−25%, such as +/−10%, or +/−5% of yield at the start of t_{P}. In these aspects, average conversion might not vary appreciably as a function of time during pyrolysis mode, and is typically within about +/−25%, such as +/−10%, or +/−5% of average conversion at the start of t_{P}.

A hydrocarbon feed is subjected to “thermal pyrolysis” when <50.0% of the heat utilized by the pyrolysis is provided by exothermically reacting the hydrocarbon feed, e.g., with an oxidant, e.g., ≤25%, such as ≤10%, or ≤1%. The “severity threshold temperature” for pyrolysis is the lowest temperature at which acetylene selectivity is at least 10% for a residence time ≤0.1 second. High-severity pyrolysis conditions are those carried out at a peak gas temperature that is greater than or equal to the severity threshold temperature. Low-severity pyrolysis conditions are those carried out at a peak gas temperature that is less than the severity threshold temperature, i.e. conditions under which substantially no hydrocarbon pyrolysis is carried out at a pyrolysis gas temperature that exceeds the severity threshold temperature. High-severity conditions include those which exhibit (i) a methane selectivity ≥5 wt. % and/or (ii) a propylene selectivity at a temperature ≥1000° C. of ≤0.6 wt. %. With respect to pyrolysis reactors, the term “residence time” means the average time duration for non-reacting (non-converting by pyrolysis) molecules (such as He, N₂, Ar) having a molecular weight in the range of 4 to 40 to traverse a pyrolysis region of a pyrolysis reactor.

The terms “reactor”, “reactor system”, “regenerator”, “recuperator”, “regenerative bed”, “monolith”, “honeycomb”, “reactant”, “fuel”, and “oxidant” have the meanings disclosed in U.S. Pat. No. 7,943,808, which is incorporated by reference herein in its entirety. A “pyrolysis reactor” is a reactor, or combination of reactors or a system for hydrocarbon pyrolysis. The term “pyrolysis stage” means at least one pyrolysis reactor, and optionally including means for conducting one or more feeds thereto and/or one or more products away therefrom. A “region” or “zone” is a location, e.g., a specific volume, within a reactor, a location between two reactors and/or the combination of different disjointed locations in one or more reactors. A “pyrolysis region” is a location where pyrolysis is carried out, e.g., in a location which contains or is proximate to components, such as at least one thermal mass, which provides heat for the pyrolysis. A reactor or reaction stage can encompass one or more reaction regions. More than one reaction can be carried out in a reactor, stage, or region.

A pyrolysis region can include components having conduits, channels, and passages. The term “conduit” refers to means for conducting a composition from one location to another. The term encompasses (i) elementary conducting means, such as a pipe or tube, and (ii) complex means such as tortuous pathways through conducting means, e.g., pipes, tubes, valves, and reactors, that are filled with random packing. The term “passage” means a geometrically contiguous volume element that can be utilized for conveying a fluid within a reactor, regenerator, recuperator, regenerative bed, monolith, honeycomb, etc. The term “channel” means a plurality of passages that can be utilized together for conveying a fluid within the reactor, regenerator, recuperator, regenerative bed, monolith, honeycomb, etc. For example, a honeycomb monolith can comprise a single channel, with the channel having a plurality of passages or sets of passages.

The term “bulk gas temperature” means the temperature of a bulk gas steam as measured by a device (such as a thermocouple) that is in contact with the bulk gas but not in contact with a solid thermal mass. For example, if the gas is traveling through an internal channel of length L_{c} of a thermal mass in the pyrolysis zone of a thermal pyrolysis reactor, the bulk gas temperature at a location along L_{c} is the average temperature (arithmetic mean) over the channel's cross sectional area at that location. The peak gas temperature is the greatest cross-sectional-averaged bulk gas temperature achieved along a flowpath. One skilled in the art will appreciate that a gas temperature immediately proximate to a solid thermal mass, such as a partition between passages within a thermal mass at any particular location may exceed the bulk gas temperature, and may, in some infinitesimal layer, actually approach the solid's temperature. The average bulk gas temperature “Tₐᵥ” over a region of the reactor, e.g., the pyrolysis zone or, a flow path, a channel, a passage, etc. Tₐᵥ at a particular time (e.g., at the start of pyrolysis) is obtained using the formula:

$$Tav = [ \frac{1}{b - a} {\int }_{a}^{b} T ( x ) dx ]$$

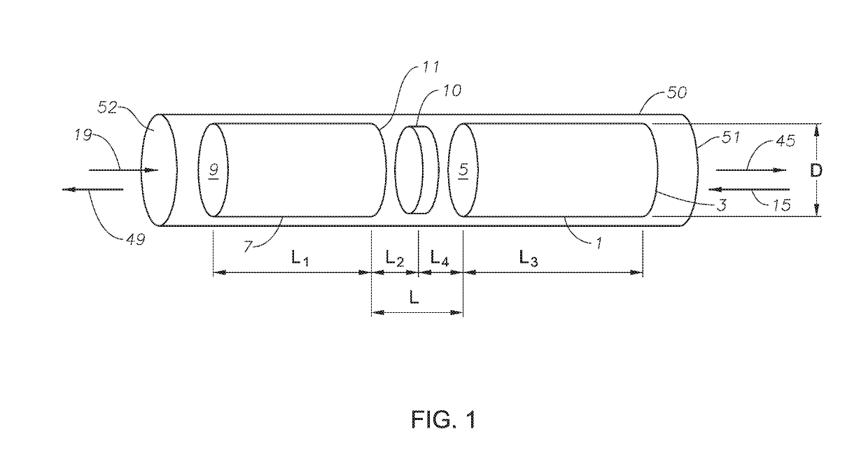

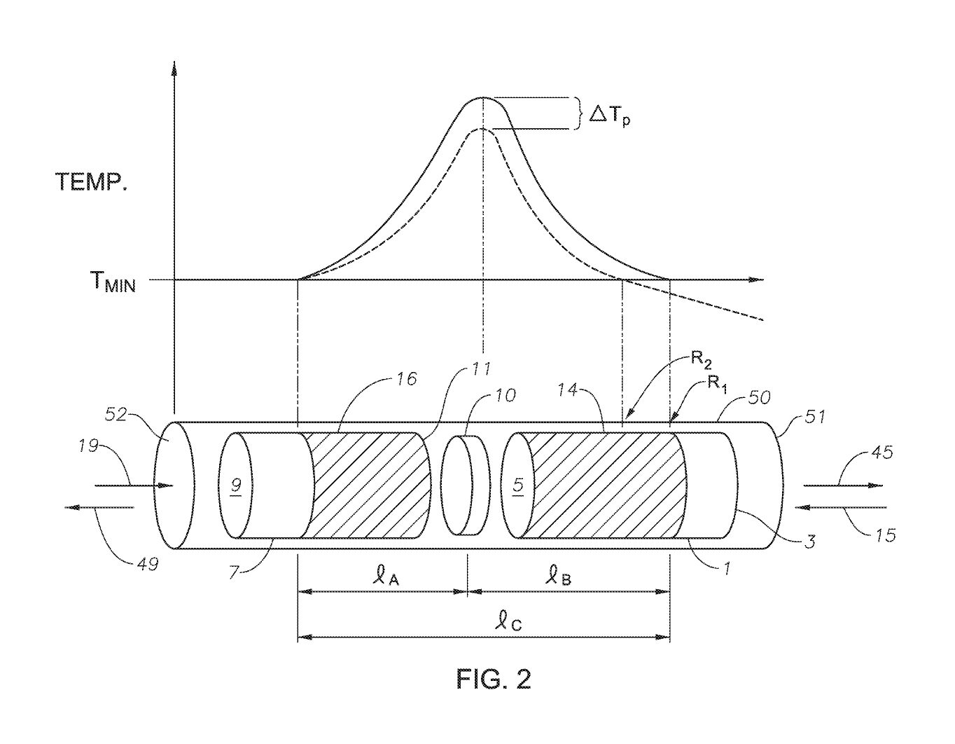

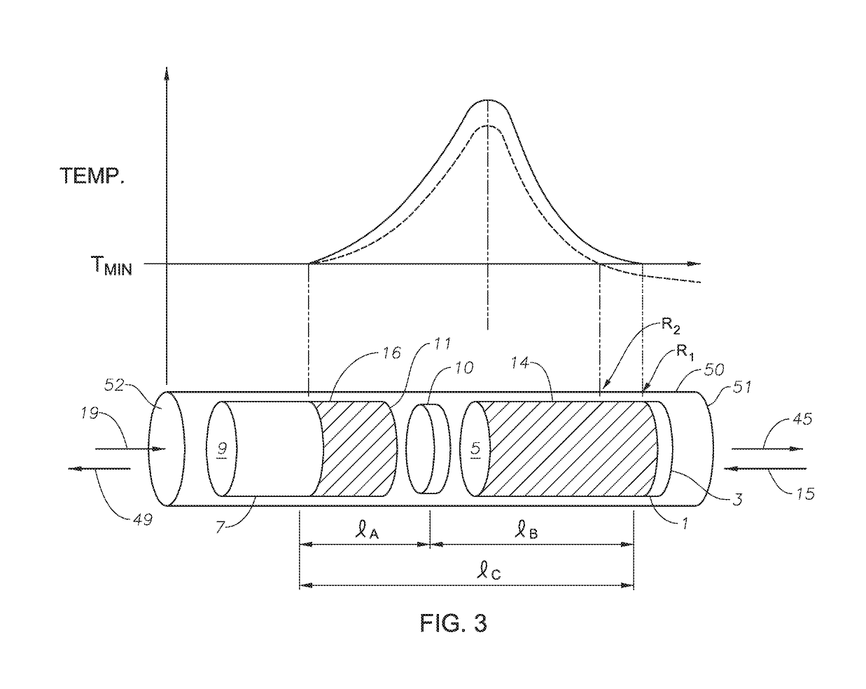

Parameters a and b are the boundaries of an interval (distance) along the long axis of the reactor. For example, referring to FIG. 1, parameter “a” can be the position of aperture 51 and parameter “b” can be the position of aperture 9. T(x) is a function representing the variation of bulk gas temperature over the interval of from a to b. When T(x) is a bulk gas temperature profile of a pyrolysis zone, e.g., the pyrolysis zones indicated (at the start of t_{P}) by the shaded regions in FIGS. 2 and 3, parameters a and b are the locations where the bulk gas temperature profile intersects the line T_{MIN}, which corresponds to the minimum temperature at which feed conversion is ≥10% under the selected pyrolysis conditions and feed. Since the bulk gas temperature profile typically changes during the pyrolysis time interval t_{P}, Tₐᵥ will typically decrease during t_{P}. The portion of the profile having a temperature ≥T_{MIN} can be continuous, but this is not required. For example, when a profile that intersects T_{MIN} at more than two locations in the pyrolysis zone (e.g., a, b) and touches T_{MIN} at a location c (not shown, but between a and b), additional integrations are carried out, e.g.:

$$Tav = \frac{1}{b - a} {\int }_{a}^{b} T ( x ) dx + \frac{1}{c - b} {\int }_{b}^{c} T ( x ) dx .$$

When the portion of the profile that is ≥T_{MIN} is in the form of discrete segments, the integrations are performed over each of the segments.

The term “Periodic Table” means the Periodic Chart of the Elements, as it appears on the inside cover of The Merck Index, Twelfth Edition. Merck & Co., Inc., 1996.

Certain aspects of the invention relate to carrying out pyrolysis mode and heating mode under the specified conditions in one or more reverse flow reactors. Representative reverse flow reactors will now be described in more detail with respect to FIG. 1. The invention is not limited to these aspects, and this description is not meant to foreclose the use of other reactors within the broader scope of the invention.

### Representative Reverse Flow Reactors

Reverse-flow reactor 50 can be in the form of an elongated tubular vessel having an internal volume which includes a pyrolysis zone for carrying out the pyrolysis. Typically, the internal volume includes three zones: a first heat-transfer zone, a second heat transfer zone, with the pyrolysis zone being located between the first and second heat transfer zones. The zones are in fluidic communication with one another. The reactor vessel's cross sectional shape and/or cross sectional area can be substantially uniform over the length of the reactor, but this is not required. For example, one or more segments of the reactor vessel's length can have a circular, elliptical, or polygonal cross section. Reactor 50 has opposed first and second openings 51 and 52 which are in fluidic communication with the internal volume and are located at terminal ends of the reactor vessel.

The reactor 50 includes first and second thermal masses 1 and 7 for transferring heat to/from reactants and products during the pyrolysis and heating modes. Typically, a first segment of the first thermal mass 1 is located in the first heat transfer zone, with a second segment being located in the pyrolysis zone. Likewise, a first segment of the second thermal mass can be located in the second heat transfer zone, with a second segment being located in the pyrolysis zone. Typically, the thermal mass is in the form of a channeled tubular member comprising bedding or packing material that is effective in storing and transferring heat, such as glass or ceramic beads or spheres, metal beads or spheres, ceramic (e.g., ceramics, which may include alumina, yttria, and zirconia) or honeycomb materials comprising ceramic and/or metal, other forms of tubes comprising ceramic and/or metal, extruded monoliths and the like. Typically, the thermal mass has an open frontal area ≤55%. Those skilled in the art will appreciate that the compositions of the first and second thermal masses should be selected from among those that substantially maintain integrity (structural and compositions) and functionality during long term exposure to pyrolysis feeds, products, and reaction conditions, e.g., temperatures ≥750° C., e.g., ≥1200° C., or for increased operating margin ≥1500° C. Regenerative beds, such as those described in U.S. Pat. No. 8,754,276 (incorporated by reference herein in its entirety) are suitable for use as thermal mass, but the invention is not limited thereto. Typically, thermal masses 1 and 7 have the form of an elongated refractory tube having (i) at least one internal channel for conveying the feeds and products through the pyrolysis zone and (ii) opposed apertures in fluidic communication with the internal channel(s). Thermal mass 1 has a length L₃ (typically L₃ is the length of the internal channel, L_{M}) and thermal mass 7 has a length L₁. L₃ is ≥0.1*L_{R}, e.g., in the range of from 0.1*L_{R} to 0.9*L_{R} such as 0.1*L_{R} to 0.4*L_{R}. Optionally, L₃ is of substantially the same length as L₁, and is of substantially the same cross-sectional shape and of substantially the same cross sectional area. As shown in FIG. 1, thermal mass 1 includes first and second apertures 3 and 5, and thermal mass 7 includes first and second apertures 9 and 11. Typically, aperture 3 is adjacent to opening 51. Optionally, particularly in aspects (not shown) in which thermal mass 7 is omitted, aperture 5 can be adjacent to opening 52. Turning again to the aspects illustrated in FIG. 1, thermal masses 1 and 7 each have the form of an elongated honeycomb comprising at least one channel, the channel having a plurality of passages. When a thermal mass is a segmented thermal mass, the honeycombs can be arranged adjacent to one another (e.g., end-to-end, in series). As may be appreciated, it is desirable, e.g., to lessen reactor pressure drop, to a align passages of a honeycomb's internal channel or channels with those of neighboring honeycombs to facilitate fluidic communication through all of the segments constituting the thermal mass. Optionally, the segments are of substantially the same composition, e.g., refractory, such as refractory comprising metal oxide-containing ceramic and/or ceramics which do not contain metal oxide.

The internal volume of reactor 50 also includes a combustion zone, typically located between terminal segments of the first and second thermal masses. Although the combustion zone can occupy less than all of the region between apertures 5 and 11, it is within the scope of the invention for the combustion zone to include all of the reactor's internal volume between apertures 5 and 11, e.g., the entire length L shown in FIG. 1. Typically, however, the combustion zone is centered in the region between apertures 11 and 5, e.g., with L₂ being substantially equal to L₄. As may be appreciated, the combustion zone occupies a region of reactor 50's internal volume that is within the pyrolysis zone. However, since in the aspects illustrated in FIG. 1, a heating mode is not carried out at the same time as pyrolysis mode, appreciable combustion does not occur in the combustion zone during pyrolysis and appreciable pyrolysis does not occur in the pyrolysis zone during heating.

The combustion zone is configured for (i) mixing the fuel and a portion of the oxidant during heating mode for efficient combustion, (ii) increasing distribution uniformity over third zone's internal cross sectional area of the combustion products, unreacted oxidant, and optionally unreacted fuel, and (iii) lessening undesirable pressure-drop effects during pyrolysis mode. The combustion zone can have the form of an open volume within the internal volume of reactor 50, e.g., an open volume having a length L and substantially constant circular cross section of diameter D and cross sectional area A (not shown). As may be appreciated, an open volume having an appropriate L: A ratio will provide at least some mixing and distribution during heating mode without creating too great a pressure drop during pyrolysis mode. More typically, since it provides improved mixing and distribution and allows a lesser overall length for the combustion zone, the combustion zone includes at least one mixer-distributor apparatus 10. The mixer-distributor, which can have the form of a relatively thin member (e.g., a plate) having one or more orifices effective for carrying out the mixing and distribution during heating mode. Generally, the orifices have sufficient cross sectional area to prevent an undesirably large pressure drop across the third zone during pyrolysis mode. Conventional mixer-distributors can be used, such as those described in U.S. Patent Application Publication No. 13-0157205 A1 and U.S. Pat. No. 7,815,873 (incorporated by reference herein in their entireties), but the invention is not limited thereto. Optionally, the combustion zone contains at least one selective combustion catalyst. Suitable selective combustion catalysts are described in U.S. Pat. No. 8,754,276, but the invention is not limited thereto. When used, a fixed bed of the selective combustion catalyst can be included as a component of mixer-distributor 10, e.g., with one or more of the mixer-distributor's plate members serving as a catalyst support. When used, mixer-distributer 10 can be located at any location within the combustion zone. Typically, however, it located approximately mid-way between apertures 11 and 5. In certain aspects, however, such as those where the amount of coke deposits in thermal mass 1 exceed that of thermal mass 7, the combustion zone is shifted downstream (with respect to fuel-oxidant flow) toward thermal mass 1. The amount of shift is typically ≤25% of L, e.g., ≤20%, such as ≤10%.

The sum of lengths L₁, L, and L₃ is typically ≥90% of the total length of reactor 50 (L_{R}), e.g., as measured between openings 51 and 52. Since it is desirable to direct fuel and oxidant flows into appropriate passages of thermal mass 7 during heating mode and to direct pyrolysis feed flow into appropriate passages of thermal mass 1 during pyrolysis mode, it is typically desired to limit the internal volume between aperture 9 and opening 52 and between aperture 3 and opening 51, to that needed for convenient reactor assemble and to prevent component interference as might otherwise occur from thermal expansion during use. The pyrolysis zone, which generally encompasses all of region L, a segment of L₁, and a segment of L₃, is typically ≥10% of the total length of reactor 50, e.g., ≥15%, such as ≥20%. It is also typical for the pyrolysis zone to encompass ≤80% of the total length of reactor 50, e.g., to leave sufficient internal volume of thermal mass 1 for pre-heating the pyrolysis feed and sufficient internal volume of thermal mass 7 for quenching the pyrolysis product, e.g., ≤60%, such as ≤40%. In certain aspects, the pyrolysis zone has a length in the range of from 10% to 60% of the total length of the reactor, e.g., in the range of from 20% to 40%. The combustion zone's length L is typically ≤50% of that of the length of the pyrolysis zone, e.g., ≤40%, such as ≤30%, or ≤20%.

Values for L, L₁, L₂, L₃, L₄, and D generally depend on the pyrolysis feed used and the rate at which it is conducted into the reactor, the fuel and oxidant compositions, and the rate at which these are conducted into the reactor, etc. Although larger and small reactors are within the scope of the invention, (i) D is typically ≥1 cm, e.g., in the range of from about 1 cm to 10 m, such as 0.1 m to 7.5 m. (ii) L_{R} is typically ≥1 cm, e.g., in the range of from about 1 cm to 20 m, such as 0.1 m to 7.5 m, (iii) L is typically ≤25% of L_{R}, e.g., ≤10%, (iv) L₁ is typically ≥35% of L_{R} e.g., ≥45%, (iv) L₃ is typically ≤35% of L_{R}, e.g., ≥45%, L₃ being optionally of substantially the same size and shape as L₁, and (v) L₂ is typically within about +/−25% of L₄, e.g., +/−10%, such as +/−5%. Aperture 3 is typically adjacent to opening 51, and aperture 9 is typically adjacent to opening 52. For example, the spacing between aperture 3 and opening 5 can be ≥0.1*L_{R}, such as ≥0.01 L_{R}, or ≥0.01*L_{R}. These spacing ranges also typically apply to the spacing between aperture 9 and opening 52.

In certain aspects (not shown) at least a portion of the fuel-oxidant combustion is carried out in a location other than within the internal volume of reactor 50. For example, fuel combustion can be carried out at a location external to reactor 50, with the combustion products, unreacted oxidant, and optionally unreacted fuel being conveyed to the vicinity of the pyrolysis zone for (i) heating the pyrolysis zone to provide a desired temperature profile for efficiently carrying out the pyrolysis and (ii) combusting catalyst coke deposits with at least a portion of the unreacted oxidant.

In aspects illustrated schematically in FIG. 1, reactor 50 is heated during heating mode by conveying a heating mixture 19 comprising fuel and oxidant through opening 52, through aperture 9 of thermal mass 7, and out of aperture 11 toward mixer-distributor 10. Typically, the fuel and oxidant are conveyed separately through different channels of thermal mass 7, and are combined to form the heating mixture downstream of thermal mass 7. Typically fuel and oxidant are heated by a transfer of heat from thermal mass 7 as the fuel and oxidant are conveyed through the channels of thermal mass 7. Combustion of the fuel and oxidant produces a combustion product. Combustion product, any un-combusted oxidant, and any un-combusted fuel enter aperture 5. When there is un-combusted oxidant in thermal mass 1, this can react with coke deposits and any un-combusted fuel to produce additional combustion product. An aggregated combustion product 45 is conducted out of aperture 3 and away from the reactor via opening 51. The aggregate combustion product typically comprises the combustion product produced in combustion zone 10, additional combustion product, typically from combustion of coke in passages of thermal mass 1, and any unreacted fuel and/or any unreacted oxidant. Reactor 50 is switched from heating mode to pyrolysis mode after achieving the desired reactor temperature profile.

Continuing with reference to FIG. 1, a pyrolysis feed 15 is conducted into reactor 50 during pyrolysis mode via opening 51. The pyrolysis feed is preheated in an upstream segment of thermal mass 1 and is typically pyrolysed in (i) a downstream segment of thermal mass 1, and optionally also in (ii) the region between thermal mass 1 and thermal mass 7, and (iii) in an upstream segment of thermal mass 7. A volatile portion 49 (typically gaseous) of the pyrolysis product is cooled in a downstream segment of thermal mass 7, and is conducted away from thermal mass 7 via aperture 9, and is conducted away from reactor 50 via opening 52. A non-volatile portion of the pyrolysis product remains in the reactor, typically as coke deposits.

Heating mode is carried out for a time duration t_{H} to achieve a desired temperature profile in the internal volume of reactor 50 for the start of pyrolysis mode, primarily by fuel-oxidant combustion in the combustion zone, coke-oxidant combustion in passages of thermal masses 1 and 7, and optionally additional fuel-oxidant combustion in internal passages of thermal mass 1 and (less typically) thermal mass 7. Pyrolysis mode is carried out for a time interval t_{P}. Pyrolysis is endothermic, and, consequently, the bulk gas temperature profile of reactor 50 transformed over the course of time interval t_{P} to one that is not appropriate for efficient pyrolysis. Reactor 50 is then switched from pyrolysis mode to heating mode to reheat the reactor, so that the desired bulk gas temperature profile is exhibited at the start of a following pyrolysis mode. Typically, valve means, e.g., a plurality of valves, are provided to (i) establish forward flows of the pyrolysis feed and the pyrolysis product during pyrolysis mode for a time duration t_{P} and (ii) establish reverse flows of the fuel, the oxidant, and the combustion product during heating mode for a time duration t_{H}. The valve means can be operated by one or more flow controllers.

Pyrolysis mode and heating mode are typically repeated in sequence, for semi-continuous or continuous operation. Intervening steps between successive pyrolysis and heating modes, e.g., one or more steps for admitting a forward or reverse flow of sweep gas to the reverse-flow reactor, can be carried out between pyrolysis mode and heating mode operation, and vice versa. Continuous or semi-continuous operation can be characterized by a “cycle time”, which constitutes the time duration from the start of a pyrolysis mode to the start of the next pyrolysis mode in the sequence, and includes the time duration of heating mode(s) and any intervening steps (when used). Cycle time can be substantially constant over a plurality of repeated cycles, but this is not required. The invention is typically practiced with relatively short cycle times compared to that of conventional processes for pyrolysing similar feed hydrocarbon at a peak pyrolysis temperature ≤1200° C. For example, cycle can be ≤60 seconds, e.g., ≤30 seconds, such as ≤15 seconds, or ≤5 seconds, or ≤1 second, or ≤0.1 second. Typically, cycle time is in the range of from 0.1 second to 60 seconds. e.g., 0.1 second to 15 seconds, such as 0.1 second to 10 seconds, or 0.1 second to 5 seconds, or 0.1 second to 1 second. When (i) the pyrolysis feed is introduced into the reactor in an average flow direction that is substantially opposite to the average flow direction of fuel and oxidant flow and/or (ii) when the flow of pyrolysis product away from the reactor is substantially opposite to the average flow direction of combustion product flow, the reactor is called a reverse-flow reactor.

Certain aspects of heating mode operation, during which reactor 50 is preheated for initial pyrolysis mode operation, or reheated for continued pyrolysis mode operation, will now be described in more detail. The invention is not limited to these aspects, and this description is not meant to foreclose other ways to operate a heating mode within the broader scope of the invention.

### Representative Heating Mode Conditions

Operating conditions during heating mode are selected to accomplish (i) reheating the pyrolysis zone to establish a temperature profile in the reactor corresponding to the desired bulk gas temperature profile at the start of a following pyrolysis mode and (ii) removing sufficient coke deposits from within the reactor's internal volume, which would otherwise lead to an increase in reactor pressure drop. When it is desired to quench the pyrolysis product within the reactor, heating mode optionally includes cooling thermal mass within the reactor at a location that is both upstream (with respect to fuel-oxidant flow) of the combustion zone and downstream (with respect to the flow of pyrolysis product) of the pyrolysis zone.

Combustion is carried out during heating mode by reacting fuel and oxidant, e.g., fuel and oxidant contained in a heating mixture. The fuel and oxidant can be the same as those disclosed in U.S. Pat. No. 7,943,808. Optionally, the fuel is derived from, comprises, consists essentially of, or consists of one or more of hydrogen, CO, methane, methane containing streams, such as coal bed methane, biogas, associated gas, natural gas and mixtures or components thereof, etc. The fuel typically comprises one or more of molecular hydrogen, synthesis gas (mixtures of CO and H₂), and hydrocarbon, such as ≤10.0 wt. % hydrocarbon, or ≥50.0 wt. % hydrocarbon, or ≥90.0 wt. % hydrocarbon. The oxidant is typically one or more of molecular oxygen, ozone, and air, including molecular oxygen in air. Those skilled in the art will appreciate that feed flow rate will depend on factors such as feed composition, reactor volume, pyrolysis conditions, etc. Accordingly, the invention can be carried out over a very wide range of heating mixture flow rates. e.g., at a flow rate ≥0.001 kg/s, such as ≥0.1 kg/s, or ≥10 kg/s, or ≥100 kg/s, or more.

Once a fuel of the desired caloric content (heating value) has been selected, the amounts of fuel and oxidant to the reactor during heating mode can be specified in terms of the amount of oxidant needed for combusting the accumulated coke deposits (“OCₐ”) and the amount of oxidant (“OC_{b}”) needed for the substantially stoichiometric combustion of the fuel. Typically, the amount of oxidant supplied during heating mode is Z·(OCₐ+OC_{b}), wherein Z is generally ≥0.5, e.g., ≥0.8, such as in the range of 0.5 to 5.0, or 0.5 to 3.0, or 0.8 to 3.0. The amounts OCₐ and OC_{b} are on a molar basis. When Z>1.0, the excess oxidant can be utilized, e.g., for one or more of removing at least a portion of any accumulated coke deposits, moderating the reaction temperature during heating mode (as disclosed in U.S. Pat. No. 7,943,808), and conveying heat within the reactor from one zone to another. Generally, a first portion of the oxidant is combusted with the fuel in the combustion zone, and a second portion is combusted with accumulated coke deposits. Typically, the first portion comprises ≥50 wt. % of the total amount of oxidant supplied during heating mode, e.g., ≥75 wt. %, or ≥90 wt. %, with the second portion comprising at least 75 wt. % of the remainder of the total oxidant, e.g., ≥90 wt. %. It is also typical for oxidant flow rate and fuel flow rate to remain substantially constant for the duration of heating mode. These flow rates are selected to achieve the desired amount of combustion heating and the desired amount of coke removal during t_{H}. The invention is compatible with conventional methods for lessening coke accumulation in thermal masses during heating mode, e.g., those described in U.S. Pat. No. 9,187,382, which is incorporated by reference in its entirety.

Other streams can be provided to the reactor during heating mode, e.g., one or more diluent streams can be provided, such as by addition to the heating mixture. When used, diluent can be provided with the oxidant and/or fuel. Suitable diluents (which can be a diluent mixture) include one or more of, e.g., oxygenate (water, carbon dioxide, etc.), non-combustible species, such as molecular nitrogen (N₂), and fuel impurities, such as hydrogen sulfide. For example, the oxidant can comprise 60.0 mole % to 95.0 mole % diluent and 5.0 mole % to 30.0 mole % molecular oxygen per mole of the oxidant, such as when the oxidant is air. Optionally, the oxidant has a mass ratio of diluent to molecular oxygen in the range of 0.5 to 20.0, e.g., in the range of 4.0 to 12.0.

In order to lessen or prevent the occurrence of a sharp temperature gradient in the bulk gas temperature profile at the start of pyrolysis mode and during the course of pyrolysis mode, it was expected that a relatively long-duration t_{H} would be needed, e.g., a t_{H} of ≤30 seconds, or ≤50 seconds. Surprisingly, this is not the case: a t_{H}≤27 seconds is typically sufficient for reheating the reactor to achieve the desired bulk gas temperature profile at the start of pyrolysis mode, e.g., ≤25 second, such as ≤10 seconds, or ≤1 second, or ≤0.1 second. For example, t_{H} can be in the range of from 0.01 second to 25 seconds, or 0.05 second to 10 seconds, or 0.05 second to 5 seconds, or 0.05 second to 1 second.

It was also expected that fuel-oxidant combustion should be distributed through the reactor's pyrolysis zone to achieve the desired non-constant bulk gas temperature profile in the pyrolysis zone during t_{P}, and to lessen or prevent the occurrence of a sharp temperature gradient in the bulk gas temperature profile during t_{P}. Surprisingly, it has been found that this is not the case. The desired bulk gas temperature profile for pyrolysis mode is established during heating mode by carrying out fuel-oxidant combustion primarily in the central region of the reactor (e.g., a region of length L as shown in FIG. 1). While not wishing to be bound by any theory model, it is believed that concentrating combustion in the central region of the reactor leads to an improved reactor temperature compared to that which is achieved by distributed combustion for mainly two reasons. First, the greater fuel and oxidant flow rates needed to achieve the desired amount of combustion during t_{H}, and the resulting increased flow rate of combustion product, leads to more favorable distribution of combustion heat within the reactor. Second, during heating mode the combination of radiative heat transfer to a thermal mass proximate to the combustion zone and heat conduction within the thermal mass sufficiently moderates the reactor temperature profile so as to broaden temperature gradients in the pyrolysis zone that would otherwise be undesirably sharp.

Referring again to FIG. 1, an appropriate combustion zone of length L can be achieved by conventional methods, e.g., by use of one or more mixer-distributors 10, use of a selective combustion catalyst, etc. For example, it has been found that even when mixer-distributors and selective combustion catalysts are not used, limiting Z to a value ≤5.0, e.g., ≤3.0, and especially <2.0, results in a combustion zone length L that is ≤50% of that of the length of the pyrolysis zone, e.g., ≤40%, such as ≤30%, or ≤20%.

After the reactor is sufficiently reheated to establish the reactor temperature profile needed for the start of pyrolysis, heating mode operation is lessened or terminated, typically by decreasing or terminating fuel and oxidant flow into the reactor. Pyrolysis mode operation in then increased or commenced by introducing a pyrolysis feed into the reactor. Representative pyrolysis feeds will now be described in more detail. The invention is not limited to these pyrolysis feeds, and this description is not meant to foreclose the use of other pyrolysis feeds within the broader scope of the invention.

### Representative Pyrolysis Feeds

The pyrolysis feed comprises C₂₊ hydrocarbon. e.g., ≥1 wt. % of C₂₊ hydrocarbon, such as ≥10 wt. %, or ≥25 wt. %, or ≤50 wt. %, or ≥75 wt. %, or ≤90 wt. %. Typically ≥90 wt. % of the remainder of the pyrolysis feed comprises diluent, e.g., one or more of methane, CO₂, water, etc. In certain aspects, the pyrolysis feed consists essentially of or even consists of C₂₊ hydrocarbon. The pyrolysis feed's hydrocarbon (the “feed hydrocarbon”) generally includes any hydrocarbon compounds or mixture of hydrocarbon compounds that when subjected to the specified pyrolysis conditions produce the desired pyrolysis product. Suitable pyrolysis feeds include those disclosed in U.S. Patent Application Publication No. 2016-176781, which is incorporated by reference herein in its entirety. In certain aspects, particularly those aspects where the feed comprises ≤50 wt. % ethane (or propane, or a mixture of ethane and propane). e.g., ≥75 wt. %, such as ≥90 wt. %, conversion during pyrolysis is based on the amount of C₂₊ hydrocarbon that is converted. In other aspects, e.g., those where the feed includes components such as (i) saturated C₄₊ hydrocarbon and/or (ii) aromatic and/or non-aromatic cores having one or more substantially-saturated C₂₊ side chains, the conversion is based on the aggregate amount of C₂₊ hydrocarbon components converted, including such substantially saturated side chains as may be converted.

Although the feed hydrocarbon typically includes C₂₊ compounds which contain hydrogen and carbon only, feed hydrocarbon compounds having covalently-bound and/or non-covalently-bound heteroatoms are within the scope of the invention. When present in the feed hydrocarbon, the amount of such heteroatom-containing hydrocarbon compounds is typically ≤10 wt. % based on the weight of the feed's hydrocarbon. Feed hydrocarbon that is substantially-free of non-aliphatic hydrocarbon is within the scope of the invention, as is feed hydrocarbon that is substantially free of aromatic hydrocarbon and/or substantially free of olefinic hydrocarbon, particularly C₂-C₅ olefinic hydrocarbon. Substantially-free in this context means <1 wt. % based on the weight of the feed hydrocarbon, such as ≤0.1 wt. %, or ≤0.01 wt. %, or ≤0.001 wt. %.

The feed hydrocarbon can be obtained from one or more sources of hydrocarbon, e.g., from natural hydrocarbon sources including those associated with producing petroleum, or from one or more synthetic hydrocarbons sources such as catalytic and/or non-catalytic reactions. Examples of such reactions include catalytic cracking, catalytic reforming, coking, steam cracking, etc. Synthetic hydrocarbon sources include those in which hydrocarbon within a geological formation has been purposefully subjected to one or more chemical transformations. The feed can include recycle components, e.g., a portion of the pyrolysis product. Such recycle, when used, can include, e.g., methane, molecular hydrogen, and C₂₊ hydrocarbon, typically C₂ to C₅ hydrocarbon.

The feed hydrocarbon can include one or more of ethane, propane, butanes, saturated and unsaturated C₆ hydrocarbon, including those derived from one or more of Fischer-Tropsch synthesis products, shale gas, biogas, associated gas, natural gas and mixtures or components thereof, steam cracked gas oil and residues, gas oils, heating oil, jet fuel, diesel, kerosene, gasoline, naphtha (including coker naphtha, steam cracked naphtha, and catalytically cracked naphtha), hydrocrackate, reformate, raffinate reformate, Fischer-Tropsch liquids, natural gasoline, distillate, virgin naphtha, crude oil, atmospheric pipestill bottoms, vacuum pipestill streams including bottoms, wide boiling range naphtha to gas oil condensates, heavy non-virgin hydrocarbon streams from refineries, vacuum gas oils, heavy gas oil, naphtha contaminated with crude, synthetic crudes, shale oils, coal liquefaction products, coal tars, tars, atmospheric resid, heavy residuum, C₄—residue admixture, naphtha—residue admixture, cracked feed, coker distillate streams, and hydrocarbon streams derived from plant or animal matter. The feed hydrocarbon can comprise volatile and non-volatile hydrocarbon, as described in U.S. Patent Application Publication No. 2016/176781. Those skilled in the art will appreciate that feed flow rate will depend on factors such as feed composition, reactor volume, pyrolysis conditions, etc. Accordingly, the invention can be carried out over a very wide range of feed flow rates. e.g., at a flow rate ≤0.001 kg's, such as ≥0.1 kg/s, or ≥10 kg/s, or ≥100 kg/s, or more.

Although the invention is not limited thereto, the specified process can be used for upgrading relatively refractory hydrocarbon, such as ethane. Accordingly, the feed hydrocarbon can comprise ethane in an amount ≥1 wt. %, e.g., ≥5 wt. %, such as ≥10 wt. %. Suitable feeds include those comprising >50 wt. % ethane, such as ≥75 wt. %, or ≥90 wt. %, or ≥95 wt. %. For example, the feed can comprise an amount of ethane in the range of from 1 wt. % to 99 wt. %, such as 5 wt. % to 95 wt. %, or 10 wt. % to 90 wt. %. One representative feed hydrocarbon comprises (i) ≥10 wt. % ethane, or ≥50 wt. %, or ≥90 wt. %, such as in the range of from 10 wt. % to 99.5 wt. % ethane, with ≥95 wt. % of the balance of the feed hydrocarbon comprising one or more of methane, propane, and butanes. In other aspects, the feed comprises ≥90 wt. % of (i) ethane and/or (ii) propane. The light paraffinic hydrocarbon can be provided from any convenient source, e.g., from synthetic and/or natural sources. Light paraffinic hydrocarbon can be provided from petroleum or petrochemical processes and/or sources of geological origin, e.g., natural gas. In particular aspects, the pyrolysis feed comprises ≥90 wt. % of (i) ethane and/or (ii) propane.

The pyrolysis feed optionally includes diluent, typically comprising compositions that are essentially non-reactive under the specified pyrolysis conditions. Examples of useful diluents include, but are not limited to, one or more of methane, water (e.g., steam), hydrogen, nitrogen and the noble gases, such as helium, neon and argon. Diluent present in the pyrolysis feed's source (e.g., methane and/or CO₂ present in natural gas) and diluent added to the pyrolysis feed are within the scope of the invention. Diluent, when present, is typically included in the pyrolysis feed in an amount ≤60 wt. % based on the weight of the feed, e.g., ≤50 wt. %, such as ≤40 wt. %, or ≤30 wt. %, or ≤20 wt. %, or ≤10 wt. %, or in the range of from 1 wt. % to 50 wt. %. Diluent is also suitable for use as a sweep gas, e.g., for (i) removing at least a portion of any deposits in the reactor after the pyrolysis mode and/or after heating mode and/or (ii) adjusting the reactor's temperature profile-heat carried by the sweep gas from warmer regions of the reactor for transfer to cooler regions will increase the temperature of the cooler regions and further lessen or prevent sharp gradients in the reactor temperature profile.

A flow of the pyrolysis feed is conducted to the pyrolysis reactor during pyrolysis mode, typically in a reverse-flow direction, e.g., one that is opposed to that of oxidant flow. During pyrolysis mode, at least a portion of the feed hydrocarbon is pyrolysed to produce a desired pyrolysis product. Certain pyrolysis conditions that are useful for pyrolysing the specified pyrolysis feeds will now be described in more detail. The invention is not limited to these pyrolysis conditions, and this description is not meant to foreclose the use of other pyrolysis conditions within the broader scope of the invention.

### Representative Pyrolysis Mode Conditions

Pyrolysis mode is operated for a time duration t_{P}. At the start of pyrolysis mode and during t_{P}, the reactor's pyrolysis zone exhibits a bulk gas temperature profile that results in a range of light olefin yields, typically without excessive coke yield. When heating mode is carried out under the specified conditions, the bulk gas temperature profile at the start of pyrolysis mode continuously varies over the length of the pyrolysis zone. Although the peak gas temperature in the pyrolysis zone decreases during the course of the pyrolysis, its position along the length of the pyrolysis zone and the general shape of the bulk gas temperature profile typically remain substantially the same during t_{P}. Regions of substantially-constant temperature along the length of the pyrolysis zone are therefore avoided. Sharp gradients in the bulk gas temperature profile within the pyrolysis zone are also substantially avoided. Although the pyrolysis can be carried out at high severity, typically it is carried out at low severity.

In order to substantially maintain the shape of the bulk gas temperature profile as the peak gas temperature decreases during pyrolysis mode, the duration of pyrolysis mode t_{P} is kept relatively short as compared to conventional reverse-flow pyrolysis reactors pyrolysing substantially similar pyrolysis feeds, particularly at a Tₐᵥ≤1400° C. e.g., ≤1200° C. In certain aspects, the duration of pyrolysis mode t_{P} is ≤20 seconds, e.g., ≤15 second, such as ≤10 seconds, or ≤1 second, or ≤0.1 second, or ≤0.01 second. For example, t_{P} can be in the range of from 1×10⁻³ second to 20 seconds, e.g., 0.01 second to 15 seconds, such as 0.05 second to 5 seconds, or 0.05 second to 1 second, or 0.05 second to 0.5 second. Conventional methods can be used to achieve these ranges of t_{P}, e.g., using one or more poppet valves and/or hydrodynamic valving, but the invention is not limited thereto. Using the specified ranges of t_{P} avoids the appearance of pyrolysis zone segments having a substantially-constant bulk gas temperature profile. Using these t_{P} values also substantially prevents relatively sharp temperature gradients in the pyrolysis zone. For example, at any time during the pyrolysis variations in the bulk gas temperature are typically ≤140° C. within any pyrolysis zone segment having a length ≤10% of I_{C}, e.g., ≤100° C. such as ≤50° C. More particularly, using these t_{P} values typically limits temperature variations to ≤75° C. within any segment of thermal mass 1 that has a length ≤10% of I_{B}, e.g., ≤50° C., such as ≤25° C.

FIG. 2 schematically shows a representative regenerative reverse-flow reactor, similar to that shown in FIG. 1, and a representative gas temperature profile. The solid line represents the bulk gas temperature profile at the start of pyrolysis mode, and the dashed line represents the bulk gas temperature profile at the end of t_{P}. At the start of pyrolysis mode, the flow of combustion mixture 19 is curtailed a flow of pyrolysis feed 15 is established. The reactor's pyrolysis zone encompasses the combustion zone, the shaded region 16 of thermal mass 7, and the shaded region 14 of thermal mass 1. Particularly at relatively large flow rates of fuel and/or oxidant during heating mode, and/or when t_{H} is of relatively long duration, the peak gas temperature Tₚ during the pyrolysis can be displaced away from the reactor's center. In such aspects, the length of the pyrolysis zone's downstream segment I_{A} is less than that of the upstream segment I_{B}, e.g., at least 10% less, such as at least 25% less, or at least 50% less. The total length of the pyrolysis zone I_{C} is the sum of I_{A} and I_{B}. Typically, I_{C} is in the range of from 10% to 50% of the total length of reactor 50. e.g., in the range of 20% to 40%. For example, I_{C} can be in the range of from 20% to 40% of L₁+L₂+L₃+L₄. The location of the terminal ends of I_{A} and I_{B} is determined by the minimum temperature T_{MIN} needed for appreciable pyrolysis of the selected pyrolysis feed under the specified pyrolysis conditions, e.g., in the range of about 500° C. to about 950° C.

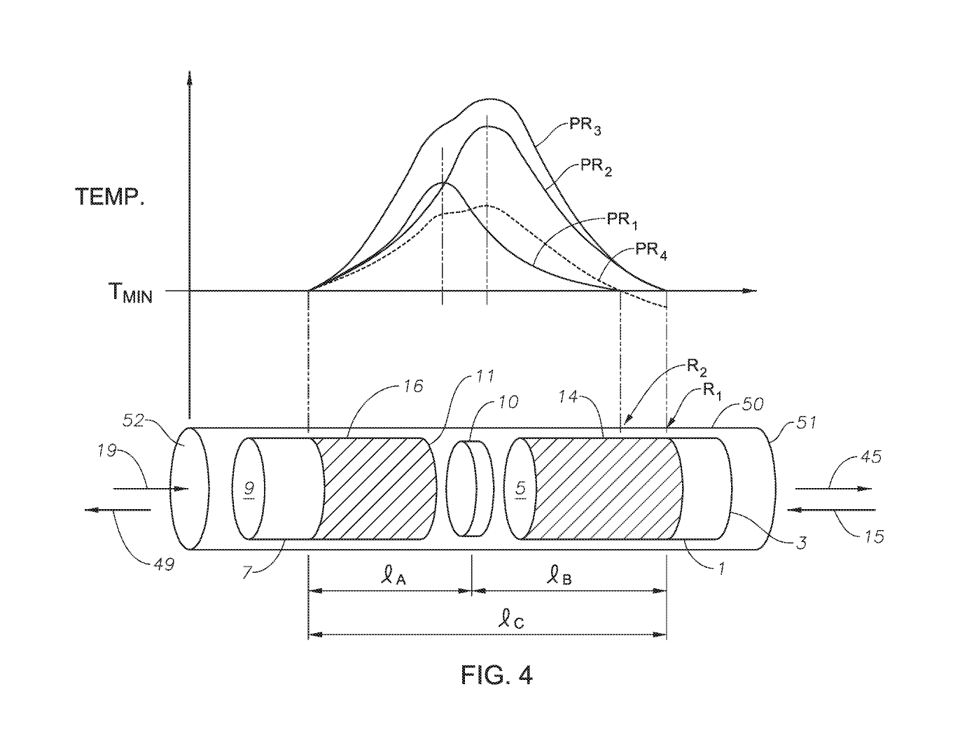

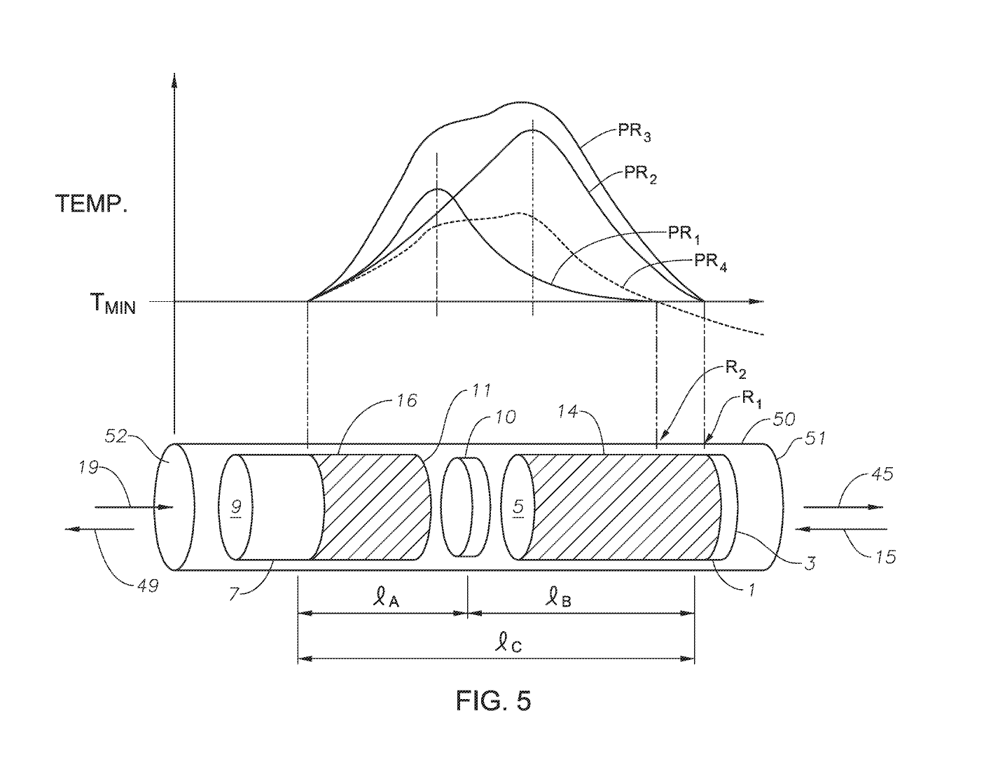

FIG. 2 shows aspects having a slight displacement of Tₚ away from the reactor center. FIG. 3 shows an even greater displacement, with Tₚ being located within shaded region 14 of thermal mass 1. Other aspects of the invention are schematically illustrated in FIG. 4. In these aspects, a mixer-distributor 10 is located within the combustion zone. In such aspects, the bulk gas temperature profile at the start of pyrolysis mode (profile PR₃) exhibits at least two local maxima, as does profile PR₄ which represents the bulk gas temperature profile at the end t_{P}. While not wishing to be bound by any theory or model, it is believed that the bi-modal bulk gas temperature profile results from heat radiated from the mixer distributor during heating mode toward thermal masses 1 and 7. Since the downstream end of the mixer-distributer achieves a greater temperature than its upstream end during heating mode, and since radiative heating is a relatively short-range phenomena (the inverse-square law applies), thermal mass 1 is heated more than thermal mass 7. The resultant bulk gas temperature profile PR₃ in the pyrolysis zone at the start of pyrolysis mode is therefore believed to be a substantially linear combination of bulk gas temperature profile PR₁, which is related to the heating of thermal mass 7, and bulk gas temperature profile PR₂, which is related to the heating of thermal mass 1. As in the case of aspects illustrated by FIG. 3, greater fuel-oxidant flow rates during heating mode lead to additional heating of thermal mass 1, e.g., by convective heat transfer from the combustion product, which displaces the peak temperature of profile PR₂ toward (or even into) shaded region 14. Such aspects are schematically illustrated in FIG. 5. The maximum gas temperature of profile PR₁ is typically 20% to 70% of the maximum gas temperature of profile PR₂, such as 30% to 70%. In FIGS. 1-5, components and streams performing similar functions have the same index number.

In aspects such as those illustrated in FIGS. 2 and 4, the pyrolysis conditions include a bulk gas temperature profile which at the start of the pyrolysis typically increases from a first temperature (T₁) proximate to the first aperture 3 of thermal mass 1 to a second temperature (T₂) proximate to the second aperture 5. The peak gas temperature Tₚ, located at a position that is at or downstream of face 5, is greater than T₂. Tₚ−T₂ at the start of pyrolysis is typically in the range of from 10° C. to 400° C., or 25° C. to 300° C. or 50° C. to 200° C. Typically, the location of Tₚ within the pyrolysis zone remains substantially constant during the pyrolysis. Substantially constant in this context means that the location of Tₚ changes during pyrolysis mode from its initial position by ≤+/−20% of I_{c}, e.g., ≤+1-15%, such as ≤+/−10%, or typically ≤+/−5%. T₁ is less than T₂, with T₂ at the start of pyrolysis typically being ≤1400° C. e.g., ≤1300° C., such as ≤1200° C. or ≤1100° C., or ≤1000° C. T₂−T₁ at the start of pyrolysis is typically in the range of from 50° C. to 500° C., such as from 100° C. to 400° C., or 100° C. to 300° C. In particular aspects utilizing a feed comprising ethane and/or propane, the pyrolysis conditions at the start of pyrolysis include T₁≤900° C., e.g., ≤750° C., such as ≤500° C.; T₂ in the range of from 975° C. to 1100° C., Tₚ≥1150° C., and T_{MIN}−T₁ in the range of from 10° C. to 400° C., or 25° C. to 300° C., or 50° C. to 200° C. At the start of the pyrolysis, the conversion of the feed's C₂₊ hydrocarbon exhibits a profile (not shown in FIG. 2) which increases from a first conversion (X₁) at a reference location R₁ positioned between the first and second apertures to a second conversion (X₂) proximate to the second aperture, wherein X₁ is in the range of from 25% to 85%, and X₂ is in the range of from 65% to 98%. Reference position R₁ is typically proximate to the end of the pyrolysis zone, as shown. The peak gas temperature decreases during t_{P}, but the bulk gas temperature profile typically maintains substantially the same shape as shown. Although the bulk gas temperature profile at the start of t_{P} is substantially congruent with that at the end of t_{P}, the location in the pyrolysis zone at which conversion X₁ is achieved translates during t_{P} from R₁ toward aperture 5 to reference position R₂ at the end of t_{P}. In particular aspects where the feed comprises ethane and/or propane, the process can include one or more of (i) X₁ in the range of from 25% to 60%, (ii) T₂ in the range of from 1025° C. to 1075° C., (iii) X₂ in the range of from 85% to 98%, (iv) the bulk gas temperature profile includes a bulk gas temperature at the reference location in the range of from 925° C. to 975° C., and (v) the reference location R₁ is positioned within 0.2*L and 0.4*L of the second aperture. More particularly, conditions at the start of the pyrolysis can include (i) an acetylene selectivity in a range of from 0% to 1% at the reference location, which acetylene selectivity increases, e.g., monotonically, to a range of 5% to 10% at the second aperture, (ii) an ethylene selectivity in a range of from 85% to 95% at the reference location, which ethylene selectivity decreases, e.g., monotonically, to a range of 70% to 85% at the second aperture, (iii) a propylene selectivity in a range of from 0.7% to 0.9% at the reference location, which propylene selectivity varies monotonically or non-monotonically to a range of 0.4% to 0.6% at the second aperture, and (iv) a butadiene selectivity in a range of from 0.5% to 1.5% at the reference location, which butadiene selectivity increases, e.g., monotonically, to a range of 4% to 5% at the second aperture.

In other aspects, shown schematically in FIGS. 3 and 5, the pyrolysis zone at time t₁ (the start of t_{P}) has a peak gas temperature Tₚ at a position located between apertures 3 and 5. Tₚ is >T₂, and T₂ is >T₁. Tₚ is typically ≤1400° C., e.g., in the range of from 1000° C. to 1400° C., such as from 1000° C. to 1300° C., or from 1025° C. to 1175° C. Tₚ−T₂ is typically in the range of from 10° C. to 400° C., or 25° C. to 300° C., or 50° C. to 200° C. T₂-T₁ is typically in the range of from 50° C. to 500° C., such as from 100° C. to 400° C. T_{MIN} is located between apertures 3 and 5. In particular aspects utilizing a feed comprising ethane and/or propane, T₁ can be ≤900° C., e.g., ≤750° C., such as ≤500° C.; with T₂ being in the range of from 975° C. to 1100° C., Tₚ being ≤1150° C., and T_{MIN}−T₁ being in the range of from 10° C. to 400° C., or 25° C. to 300° C., or 50° C. to 200° C. In aspects where the feed comprises ethane and/or propane, T₁ is typically ≤750° C. and T₂ is in the range of from 900° C. to 1100° C. As in the aspects illustrated in FIGS. 2 and 4, the aspects illustrated in FIGS. 3 and 5 exhibit a favorable conversion profile for the feed's saturated C₂₊ hydrocarbon. At t₁, the conversion profile continuously varies from a minimum conversion Xₘᵢₙ at a reference location between the first and second apertures to a second conversion (X₂) at the second aperture. Proximate to the location of Tₚ, the conversion profile exhibits a peak (Xₚ), wherein Xₚ is >X₂. At t₁, X₂ is in a range of from 55% to 95% and Xₚ is ≤98%. During t_{P}, the conversion profile (not shown) exhibits a continuously decreasing conversion at the second aperture and a continuously decreasing Xₚ. In particular aspects where the feed comprises ethane and/or propane, at the time t₁ (i) T₂ is in the range of from 925° C. to 1075° C. and (ii) X₂ is in the range of from 85% to 98%. Typical pyrolysis conditions include one or more of (A) an acetylene selectivity in a range of from 5% to 10% at the second aperture at t₁, which acetylene selectivity decreases into a range of about 0% to 1% at t₂, (B) an ethylene selectivity in a range of from 75% to 80% at the second aperture at t₁, which ethylene selectivity increases into a range of about 90% to 95% at t₂, (C) a propylene selectivity in a range of from 0.7% to 0.9% at the second aperture at t₁, which propylene selectivity varies by no more than about +/−20% during t_{P}, and (D) a butadiene selectivity in a range of from 4% to 5% at the second aperture at t₁, which butadiene selectivity decreases into a range of about 0.% to 1.5% at t₂.

In any of the foregoing aspects, the pyrolysis conditions include a feed hydrocarbon partial pressure ≤7 psia (48 kPa), or ≤10 psia (69 kPa), or ≥20 psia (138 kPa), or ≥30 psia (207 kPa). The total pressure is typically ≥5 psig (34 kPag), or ≥15 psig (103 kPag), or ≥40 psig (276 kPag), or ≥80 psig (552 kPag), or ≥120 psig (827 kPag). Particularly when the pyrolysis feed includes diluent, the total pressure can be ≥500 psig (3447 kPag), or ≥300 psig (2068 kPag), or ≥150 psig (1034 kPag). Total gas residence time in the pyrolysis zone is typically of ≤1.0 second, or ≤0.8 second; preferably ≤0.75 second or ≤0.6 second; or in the range of 0.001 second to 1.0 second, or in the range of 0.01 second to 0.8 second, or in the range of 0.01 second to 0.75 second. For example, the pyrolysis feed can be passed through thermal mass 1 at a total gas residence time at a bulk gas temperature ≥800° C. that is ≤0.100 second, such as ≤0.060 second, such as ≤0.040 second, or in the range of 0.001 second to 0.100 second, or in the range of 0.002 second to 0.060 second, or in the range of 0.002 second to 0.040 second. The pyrolysis conditions in the pyrolysis zone during t_{P} generally include Tₚ≤1400° C., Tₐᵥ≤1200° C. Typically, the pyrolysis conditions include Tₚ≤1200° C., e.g., ≤1100° C., such as ≤1000° C. or in the range of from 1000° C. to 1400° C.; and Tₐᵥ≤1100° C., e.g., ≤1000° C., such as ≤900° C., or in the range of from 900° C. to 1100° C., or 925° C. to 1075° C. During pyrolysis mode (e.g., during t_{P}), Tₚ and/or Tₐᵥ (e.g., during t_{P}) typically decrease by ≤100° C. or less, e.g., ≤75° C. such as ≤50° C. or ≤25° C., or ≤10° C., or ≤5° C. This is particularly the case when a channeled member in the pyrolysis zone has an OFA≤55%, e.g., ≤45%, such as ≤40%, or ≤30%, or in the range of from 10% to 55%, or 15% to 50%, or 20% to 45%, or 25% to 35%. Since the temperature profile during pyrolysis varies over the length of the pyrolysis zone, and typically over the entire length of the reactor, Tₚ is >Tₐᵥ. Generally, when Tₐᵥ in the channel exceeds 800° C., it is beneficial for ΔTₐᵥ to be ≤60° C., when Tₐᵥ in the channel exceeds 900° C., it is beneficial for ΔTₐᵥ to be ≤50° C., when Tₐᵥ in the channel exceeds 1000° C., it is beneficial for ΔTₐᵥ to be ≤40° C., and when Tₐᵥ in the channel exceeds 1100° C., it is beneficial for ΔTₐᵥ to be ≤20° C.

By modulating bulk gas temperature over the length of the pyrolysis zone during pyrolysis mode, the pyrolysis product conducted away from the reactor comprises a range of desired hydrocarbon products, including a desirable range of C₂-C₅ olefin. Typically, one or more of the desired hydrocarbon compounds is separated from the pyrolysis product, e.g., for storage and/or further processing. For example, one or more of ethylene, propylene, butadiene butenes, etc. can be separated from the pyrolysis product, e.g., for recovery and use in producing products such as fuels and fuel additives, oxygenates, polymer, etc. Molecular hydrogen and methane can be separated and recovered from the pyrolysis product, e.g., as a tail gas. Light paraffinic hydrocarbon can be separated recovered, e.g., for use as a fuel, such as a fuel for heating mode. Aromatic hydrocarbon, such as one or more of benzene, toluene, and xylenes, can be separated and recovered, e.g., for producing chemical and petrochemical products including fuel, solvents, polymer, etc. Conventional separations and recovery methods can be used, e.g., those described in U.S. Patent Application Publication No. 2016/176781, but the invention is not limited thereto.

Certain representative pyrolysis products will now be described in more detail. The invention is not limited to these products, and this description is not meant to foreclose the production of other pyrolysis products within the broader scope of the invention.

### Representative Pyrolysis Products

In certain aspects, the pyrolysis product conducted away from the reactor is primarily gaseous and comprises molecular hydrogen; methane; ethane; ethylene; propane; propylene; butanes; butenes; butadiene; C₅ hydrocarbon, including normal, iso, and cyclo C₅ olefin and paraffin, and C₆₊ hydrocarbon, including aromatics and normal, iso, and cyclo C₆₊ olefin and paraffin. For example, when utilizing one representative pyrolysis feed comprising light paraffinic hydrocarbon and representative heating mode and pyrolysis mode conditions, the pyrolysis product can comprise 2 wt. % to 10 wt. % methane, 50 wt. % to 95 wt. % ethylene, 0.2 wt. % to 1 wt. % propylene, 0.1 wt. % to 5 wt. % butadiene, and up to about 3 wt. % benzene, based on the weight of the pyrolysis product. As may be appreciated, these ver desirable compositional ranges for the identified hydrocarbon compounds are achieved not only at the start of pyrolysis mode, but during the duration of t_{P}. This stands in sharp contrast to conventional processes operating at a gas temperature ≤1200° C., such as steam cracking. Since the conventional processes operate with little temperature variation in the pyrolysis zone, they produce a pyrolysis product having very narrow compositional ranges for the desired hydrocarbon compounds.

### Example

In this prophetic example, a pyrolysis feed consisting essentially of ethane is exposed to the specified pyrolysis conditions in a representative reverse-flow reactor configured to be similar to the one illustrated in FIG. 2. The peak gas temperature of 1200° C. at the start of pyrolysis mode is achieved at about midway along the reactor's length (proximate to the midpoint of I_{C}). About 40% of the reactors length is in the pyrolysis zone. The bulk gas temperature profile in the pyrolysis zone, which is similar to that illustrated in FIG. 2, exhibits a bulk gas temperature of about 900° C. at position R₁ at the start of pyrolysis mode (t₁), and at R₂ at the end of pyrolysis mode (t₂). R₂ is located about midway along the length of thermal mass 1. The duration of pyrolysis mode (t=t₂−t₁) is within the specified range. Even though Tₚ and Tₐᵥ decrease during the pyrolysis, the location of Tₚ within the reactor remains substantially constant during the pyrolysis.

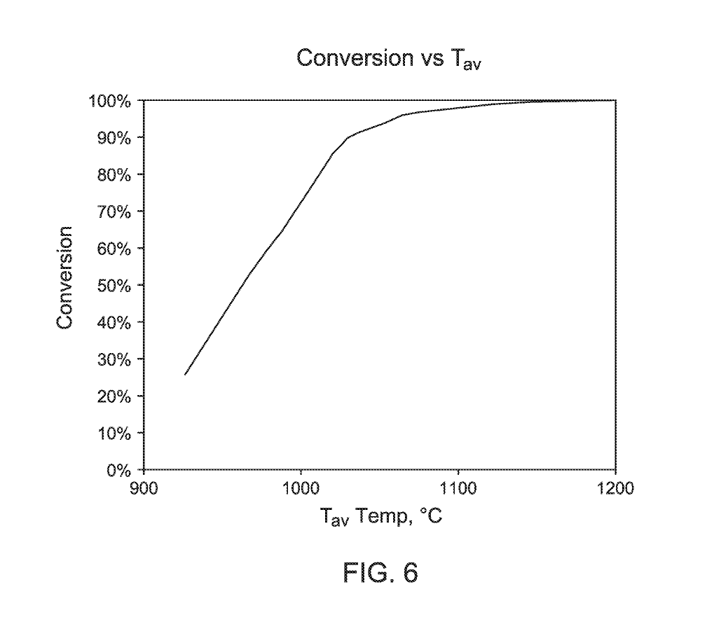

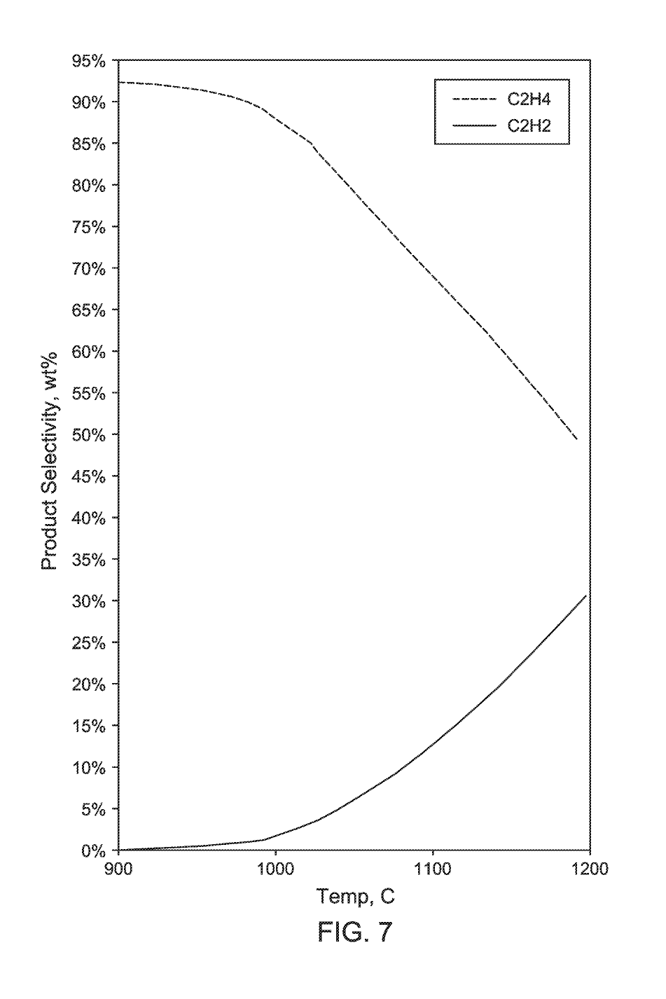

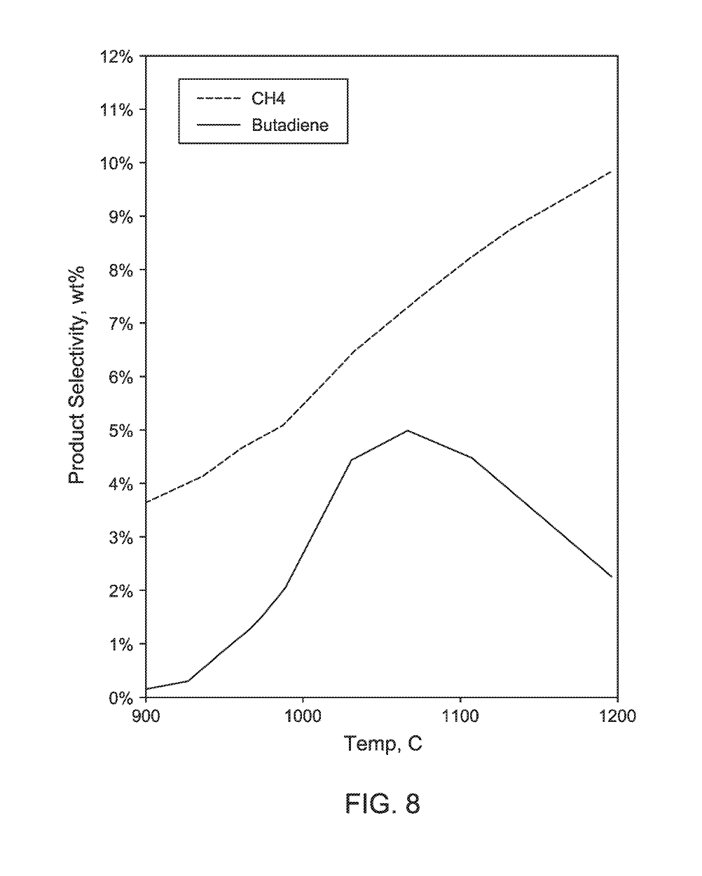

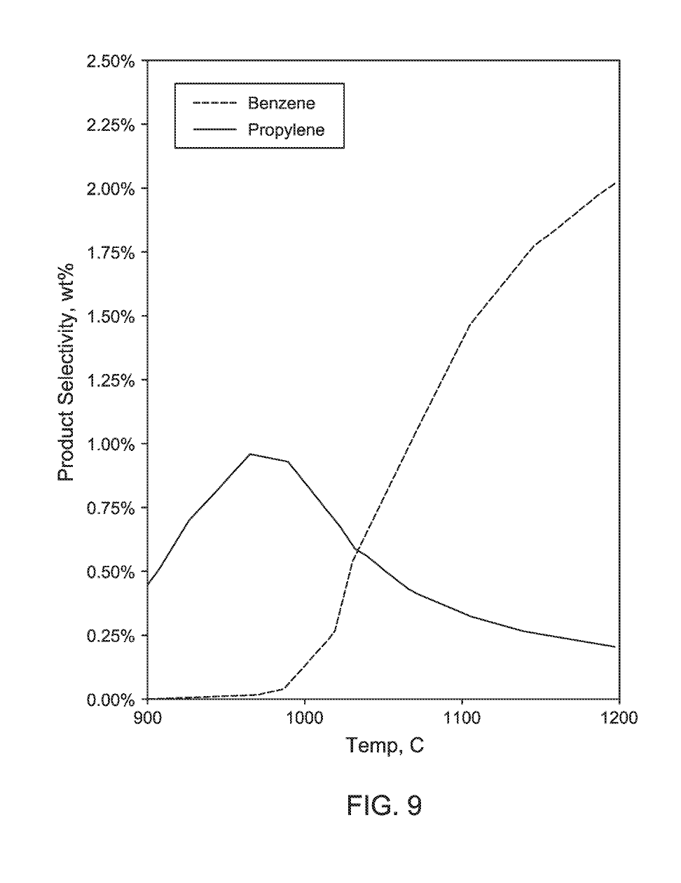

The pyrolysis product is quenched inside the reactor as it flows through the unshaded segment of thermal mass 1. FIG. 6 shows the ethane conversion achieved over a segment of the reactor from location R₁ to the midpoint of I_{C}, over which distance the temperature varies from about 925° C. to 1200° C. FIGS. 7-9 show the selectivities of the desired hydrocarbon compounds over the same segment. As shown in the figures, the conversion and desired product selectivities exhibit substantially continuous variation over the segment.

All patents, test procedures, and other documents cited herein, including priority documents, are fully incorporated by reference to the extent such disclosure is not inconsistent. It is not intended that the scope of the claims appended hereto be limited to the descriptions set forth herein but rather that the claims be construed as encompassing all the features of patentable novelty which reside herein, including all features which would be treated as equivalents thereof by those skilled in the art to which this disclosure pertains. When lower and upper limits are specified, ranges from any lower limit to any upper limit are expressly within the scope of the invention. The term “comprising” is synonymous with the term “including”. When a composition, an element or a group of components is preceded with the transitional phrase “comprising”, the same composition or group of components is within transitional phrases “consisting essentially of,” “consisting of,” “selected from the group of consisting of,” or “is” preceding the recitation of the composition, component, or components, and vice versa.

1. A hydrocarbon pyrolysis process, the process comprising:
(a) providing a feed comprising ≥1 wt. % of C₂₊ hydrocarbon;
(b) providing an elongated flow-through reactor having (i) an internal volume which includes first and second regions and (ii) opposed first and second openings in fluidic communication with the internal volume, wherein the first and second openings are separated by a reactor length (L_{R});
(c) providing at least one heated thermal mass located in the first region, wherein (i) the thermal mass includes first and second apertures and at least one internal channel, (ii) the first and second apertures are in fluidic communication with the channel, and are separated by a flow-path of length L_{M} through the channel, (iii) L_{M} is ≥0.1*L_{R}, and (iv) the first opening is proximate to the first aperture;
(d) establishing a flow of the feed in the channel toward the second aperture by introducing the feed through the first opening and through the first aperture;
(e) pyrolysing the feed flow's C₂₊ hydrocarbon in the channel under pyrolysis conditions to produce a flow of a pyrolysis product comprising molecular hydrogen, ethylene, propylene, and butadiene, the pyrolysis conditions including:
(i) a first bulk gas temperature profile at the start of the pyrolysis which increases from a first temperature (T₁) proximate to the first aperture to a second temperature (T₂) proximate to the second aperture, T₂ being in the range of from 800° C. to 1400° C.; and
(ii) a peak gas temperature (Tₚ) within the internal volume, Tₚ being located within the second region, wherein during the pyrolysis (A) the peak gas temperature decreases from a temperature that is >T₂ at the start of the pyrolysis and (B) the location of Tₚ remains substantially constant; and
(f) conducting the flow of the pyrolysis product into the second region of the internal volume via the second aperture, and away from the reactor via the second opening.

2. A hydrocarbon pyrolysis process, the process comprising:
(a) providing a feed comprising ≥1 wt. % of C₂₊ hydrocarbon;
(b) providing an elongated flow-through reactor having (i) an internal volume which includes first and second regions and (ii) opposed first and second openings in fluidic communication with the internal volume the first and second openings being separated by a reactor length (L_{R});
(c) providing at least one heated thermal mass located in the first region, wherein (i) the thermal mass includes first and second apertures and at least one internal channel, (ii) the first and second apertures are in fluidic communication with the channel, and are separated by a flow-path of length L_{M} through the channel, (iii) L_{M} is ≥0.1*L_{R}, and (iv) the first opening is proximate to the first aperture;
(d) establishing a flow of the feed in the channel toward the second aperture by introducing the feed through the first opening and through the first aperture;
(e) pyrolysing the feed flow's C₂₊ hydrocarbon in the channel under pyrolysis conditions to produce a flow of a pyrolysis product comprising molecular hydrogen, ethylene, propylene, and butadiene, the pyrolysis conditions including:
(i) a first bulk gas temperature profile at the start of the pyrolysis which increases from a first temperature (T₁) proximate to the first aperture to a second temperature (T₂) proximate to the second aperture, T₂ being in the range of from 800° C. to 1400° C.; and
(ii) a conversion profile for the feed's C₂₊ hydrocarbon which at the start of the pyrolysis increases from a first conversion (X₁) at a reference location R₁ positioned between the first and second apertures to a second conversion (X₂) proximate to the second aperture, wherein X₁ is in the range of from 25% to 85%, and X₂ is in the range of from 65% to 98%; and
(f) conducting the flow of the pyrolysis product into the second region of the internal volume via the second aperture, and away from the reactor via the second opening.

3. The process of claim 1, wherein (i) the reactor is a reverse-flow thermal pyrolysis reactor, the reactor further comprising a second thermal mass located in the second region of the internal volume, the second thermal mass having at least one internal channel in fluidic communication with the internal channel of the first thermal mass, and (ii) the process further comprises conducting the pyrolysis product through the internal channel of the second thermal mass before the pyrolysis product is conducted away from the reverse-flow reactor, and cooling the pyrolysis product by transferring heat from the pyrolysis product to the second thermal mass.

4. The process of claim 1, wherein the pyrolysis conditions at the start of the pyrolysis further include T₁≤750° C., T₂ in the range of from 975° C. to 1100° C., a partial pressure of ≥7 psia (48 kPa), a total pressure of ≥5 psig (34 kPag), and a total gas residence time in the range of from 0.01 second to 0.8 second.

5. The process of claim 1, wherein the feed comprises one or more of ethane, propane, butanes, saturated and unsaturated C₆ hydrocarbon, including those derived from one or more of Fischer-Tropsch synthesis products, shale gas, biogas, associated gas, natural gas and mixtures or components thereof, steam cracked gas oil and residues, gas oils, heating oil, jet fuel, diesel, kerosene, gasoline, naphtha (including coker naphtha, steam cracked naphtha, and catalytically cracked naphtha), hydrocrackate, reformate, raffinate reformate, Fischer-Tropsch liquids, natural gasoline, distillate, virgin naphtha, crude oil, atmospheric pipestill bottoms, vacuum pipestill streams including bottoms, wide boiling range naphtha to gas oil condensates, heavy non-virgin hydrocarbon streams from refineries, vacuum gas oils, heavy gas oil, naphtha contaminated with crude, synthetic crudes, shale oils, coal liquefaction products, coal tars, tars, atmospheric resid, heavy residuum, C₄—residue admixture, naphtha—residue admixture, cracked feed, coker distillate streams, and hydrocarbon streams derived from plant or animal matter.

6. The process of claim 2, wherein the feed comprises ≥90 wt. % of (i) ethane and/or (ii) propane.

7. The process of claim 6, wherein (i) X₁ is in the range of from 25% to 60%, (ii) T₂ is in the range of from 1025° C. to 1075° C., (iii) X₂ is in the range of from 85% to 98%, (iv) the bulk gas temperature profile includes a bulk gas temperature at the reference location in the range of from 925° C. to 975° C., and (v) the reference location R₁ is positioned within 0.2*L_{M} and 0.4*L_{M} of the second aperture.

8. The process of claim 6, wherein the pyrolysis conditions at the start of the pyrolysis further include:
(i) an acetylene selectivity in a range of from 0% to 1% at the reference location, which acetylene selectivity increases to a range of 5% to 10% at the second aperture,
(ii) an ethylene selectivity in a range of from 85% to 95% at the reference location, which ethylene selectivity decreases to a range of 70% to 85% at the second aperture,
(iii) a propylene selectivity in a range of from 0.7% to 0.9% at the reference location, which propylene selectivity varies non-monotonically to a range of 0.4% to 0.6% at the second aperture, and
(iv) a butadiene selectivity in a range of from 0.5% to 1.5% at the reference location, which butadiene selectivity increases to a range of 4% to 5% at the second aperture.

9. The process of claim 6, wherein the pyrolysis conditions further include a second bulk gas temperature profile at the end of the pyrolysis, the second bulk gas temperature profile being substantially congruent with the first bulk gas temperature profile.

10. The process of claim 6, further comprising carrying out the pyrolysis for a time duration in the range of about 1×10⁻³ seconds to 10 seconds and then terminating the pyrolysis, wherein the pyrolysis conditions further include (i) a second conversion profile at the end of the pyrolysis, (ii) the first and second conversion profiles are substantially congruent, and (iii) the conversion X₁ at the end of the pyrolysis is achieved at a second reference location R₂, with R₂ being positioned between the R₁ and second aperture.

11. A hydrocarbon pyrolysis process, the process comprising:
(a) providing a feed comprising ≥1 wt. % of C₂₊ hydrocarbon;
(b) providing an elongated flow-through reactor having (i) an internal volume which includes first and second regions and (ii) opposed first and second openings in fluidic communication with the internal volume the first and second openings being separated by a reactor length (L_{R});
(c) providing at least one heated thermal mass located in the first region, wherein (i) the thermal mass includes first and second apertures and at least one internal channel, (ii) the first and second apertures are in fluidic communication with the channel, and are separated by a flow-path of length L_{M} through the channel, (iii) L_{M} is ≥0.1*L_{R}, and (iv) the first opening is proximate to the first aperture;
(d) initiating at a time t₁ a flow of the feed in the channel toward the second aperture by introducing the feed through the first opening and through the first aperture, and terminating the flow of feed at a later time t₂, wherein the time duration t_{P} of feed flow is substantially equal to t₂−t₁,
(e) pyrolysing the feed flow's C₂₊ hydrocarbon in the channel under pyrolysis conditions during t_{P} to cool the thermal mass and produce a flow of a pyrolysis product comprising molecular hydrogen, ethylene, propylene, and butadiene, the pyrolysis conditions including:
(i) a peak gas temperature Tₚ located in the internal volume, the peak gas temperature being located along L_{M}, and
(ii) a first bulk gas temperature profile at t₁ which varies continuously along L_{M} from a first temperature (T₁) proximate to the first aperture to a second temperature (T₂) proximate to the second aperture, wherein T₁<T₂, T₂<Tₚ, and T₂ is in the range of from 800° C. to 1400° C., and
(iii) during t_{P}, Tₚ continuously decreases and the position of Tₚ along L_{M} remains substantially constant; and
(f) during t_{P}, conducting the flow of the pyrolysis product into the second region of the internal volume via the second aperture, and away from the reactor via the second opening.

12. A hydrocarbon pyrolysis process, the process comprising:
(a) providing a feed comprising ≥1 wt. % of C₂₊ hydrocarbon;
(b) providing an elongated flow-through reactor having (i) an internal volume which includes first and second regions and (ii) opposed first and second openings in fluidic communication with the internal volume the first and second openings being separated by a reactor length (L_{R});
(c) providing at least one heated thermal mass located in the first region, wherein (i) the thermal mass includes first and second apertures and at least one internal channel, (ii) the first and second apertures are in fluidic communication with the channel, and are separated by a flow-path of length L_{M} through the channel, (iii) L_{M} is ≥0.1*L_{R}, and (iv) the first opening is proximate to the first aperture;
(d) initiating at a time t₁ a flow of the feed in the channel toward the second aperture by introducing the feed through the first opening and through the first aperture, and terminating the flow of feed at a later time t₂, wherein the time duration t_{P} of feed flow is substantially equal to t₂−t₁,
(e) pyrolysing the feed flow's C₂₊ hydrocarbon in the channel under pyrolysis conditions during t_{P} to cool the thermal mass and produce a flow of a pyrolysis product comprising molecular hydrogen, ethylene, propylene, and butadiene, the pyrolysis conditions including:
(i) a first conversion profile for the feed's C₂₊ hydrocarbon at t₁ which continuously varies from a first conversion (X₁) at a reference location between the first and second apertures to a second conversion (X₂) at the second aperture and exhibits at least one peak of conversion Xₚ located between the reference location and the second aperture, wherein Xₚ>X₂, X₂>X₁, X₂ is in a range of from 55% to 95%, Xₚ continuously decreases during t_{P}, and the location of Xₚ remains substantially constant during t_{P}, and
(ii) a first bulk gas temperature profile at t₁ which continuously varies from a first temperature (T₁) proximate to the first aperture to a second temperature (T₂) proximate to the second aperture, wherein (A) the first bulk gas temperature profile at t₁ includes a peak gas temperature Tₚ at substantially the same location as Xₚ, (B) T₁<T₂, (C) T₂<Tₚ and (D) T₂ is in the range of from 800° C. to 1400° C.; and
(f) during t_{P}, conducting the flow of the pyrolysis product into the second region of the internal volume via the second aperture, and away from the reactor via the second opening.

13. The process of claim 11, wherein (i) the pyrolysis conditions further include a partial pressure of ≥7 psia (48 kPa), a total pressure of ≥5 psig (34 kPag), and a total gas residence time in the range of from 0.01 second to 0.8 second, and (ii) the time duration t_{P} is in a range of from 1×10⁻³ seconds to 10 seconds.

14. The process of claim 11, wherein (i) the pyrolysis product further comprises coke and one or more of acetylene, benzene, methane, and at least a portion of any unconverted feed, and (ii) at least a portion of the coke remains in the internal channel as a deposit.

15. The process of claim 11, wherein the feed comprises one or more of ethane, propane, butanes, saturated and unsaturated C₆ hydrocarbon, including those derived from one or more of Fischer-Tropsch synthesis products, shale gas, biogas, associated gas, natural gas and mixtures or components thereof, steam cracked gas oil and residues, gas oils, heating oil, jet fuel, diesel, kerosene, gasoline, naphtha (including coker naphtha, steam cracked naphtha, and catalytically cracked naphtha), hydrocrackate, reformate, raffinate reformate, Fischer-Tropsch liquids, natural gasoline, distillate, virgin naphtha, crude oil, atmospheric pipestill bottoms, vacuum pipestill streams including bottoms, wide boiling range naphtha to gas oil condensates, heavy non-virgin hydrocarbon streams from refineries, vacuum gas oils, heavy gas oil, naphtha contaminated with crude, synthetic crudes, shale oils, coal liquefaction products, coal tars, tars, atmospheric resid, heavy residuum, C₄—residue admixture, naphtha—residue admixture, cracked feed, coker distillate streams, and hydrocarbon streams derived from plant or animal matter.

16. The process of claim 12, wherein the feed comprises ≥90 wt. % of (i) ethane and (ii) propane.

17. The process of claim 16, wherein at the time t₁ (i) T₂ is in the range of from 925° C. to 1075° C. and (ii) X₂ is in the range of from 85% to 98%.

18. The process of claim 16, wherein the reference location is within 0.2*L_{M} and 0.4*L_{M} of the second aperture.

19. The process of claim 16, wherein the pyrolysis conditions further include:
(A) an acetylene selectivity in a range of from 5% to 10% at the second aperture at t₁, which acetylene selectivity decreases into a range of about 0% to 1% at t₂,
(B) an ethylene selectivity in a range of from 75% to 80% at the second aperture at t₁, which ethylene selectivity increases into a range of about 90% to 95% at t₂,
(C) a propylene selectivity in a range of from 0.7% to 0.9% at the second aperture at t₁, which propylene selectivity varies by no more than about +/−20% during t_{P}, and
(D) a butadiene selectivity in a range of from 4% to 5% at the second aperture at t₁, which butadiene selectivity decreases into a range of about 0% to 1.5% at t₂.

20. The process of claim 16, wherein the pyrolysis conditions further include (i) a second gas temperature profile at t₂, the second bulk gas temperature profile exhibiting a continuously decreasing gas temperature at the second aperture during t_{P} and being substantially congruent with the first bulk gas temperature profile, and (ii) a second conversion profile for the feed's C₂₊ hydrocarbon at t₂ which is substantially congruent with the first conversion profile.

21. A hydrocarbon pyrolysis process, the process comprising:
(a) providing a feed comprising gaseous C₂₊ hydrocarbon;
(b) providing an oxidant and a fuel;
(c) providing an elongated flow-through reactor having (i) an internal volume which includes first and second regions and (ii) opposed first and second openings in fluidic communication with the internal volume the first and second openings being separated by a reactor length (L_{R}), and (iii) a first temperature profile;
(d) providing at least one heated thermal mass located in the first region, wherein (i) the thermal mass includes first and second apertures and at least one internal channel, (ii) the first and second apertures are in fluidic communication with the channel, and are separated by a flow-path of length L_{M} through the channel, (iii) L_{M} is ≥0.1*L_{R}, and (iv) the first opening is proximate to the first aperture;
(e) during a first time interval having a duration t_{P}, (i) establishing a forward flow of the feed through the first opening, through the first aperture, into the into the channel, and toward the second aperture, (ii) pyrolysing the feed flow's C₂₊ hydrocarbon in the channel under low-severity pyrolysis conditions which cools the thermal mass to achieve a second thermal profile, deposits coke in the channel, and establishes a forward flow of a pyrolysis product comprising molecular hydrogen and olefin, (iii) conducting the forward flow of the pyrolysis product into the second region of the internal volume via the second aperture, and away from the reactor via the second opening; and (iv) decreasing or halting the feed flow; wherein the low-severity pyrolysis conditions include:
(A) a first bulk gas temperature profile at the start of the first time interval which increases substantially monotonically from a first temperature (T₁) proximate to the first aperture to a second temperature (T₂) proximate to the second aperture, T₂ being in the range of from 800° C. to 1400° C.; and
(B) a peak gas temperature (Tₚ) within the second region, wherein during t_{P} the peak gas temperature decreases from a temperature that is >T₂ and the location of Tₚ remains substantially constant; and
(e) during a second time interval having a duration t_{H}, (i) establishing a reverse flow of the fuel and a reverse flow of the oxidant through the second opening and into the second region of the internal volume, the oxidant flow comprising first and second portions of the oxidant, (ii) combusting the first portion of the oxidant flow under combustion conditions with at least a portion of the fuel flow in the second region to produce a reverse flow of a first combustion product toward the thermal mass, (iii) establishing a reverse flow of the first combustion product and the second portion of the oxidant into the channel at the second aperture toward the first aperture, (iv) combusting in the internal channel the second portion of the oxidant flow with at least a portion of the deposited coke to produce a reverse flow of a second combustion product, (v) conducting the reverse flows of the first and second combustion products away from the first aperture and out of the reverse-flow reactor via the first opening, wherein heat is transferred to the thermal mass from the combustion of the first and second oxidant portions to re-heat the thermal mass to substantially achieve the first temperature profile; and (vi) decreasing the reverse flow of fuel and the reverse flow of oxidant.

22. The pyrolysis product of claim 1.

23. A reverse-flow reactor, comprising:
(a) a reactor vessel having an internal volume which includes opposed first and second heat-transfer zones, a pyrolysis zone located between the first and second heat-transfer zones, and a combustion zone, wherein the pyrolysis zone, the combustion zone, the first heat transfer zone, and the second heat transfer zone are in fluidic communication;
(b) at least one feed conduit in fluidic communication with the first heat-transfer zone to convey a forward flow of a gaseous feed comprising C₂₊ hydrocarbon through the first heat transfer zone and into the pyrolysis zone, the pyrolysis zone being adapted to:
(i) pyrolyse at least a portion of the feed and produce a pyrolysis product comprising coke, molecular hydrogen, and olefin, and
(ii) establish a forward flow of the pyrolysis product out of the pyrolysis zone and through the second heat-transfer zone and deposit at least a portion of the coke in the pyrolysis zone, wherein (A) the reactor has a peak gas temperature (Tₚ) at a location within the pyrolysis zone, and (B) the location of Tₚ remains substantially constant during the pyrolysis;
(c) at least one pyrolysis product conduit in fluidic communication with the second heat-transfer zone to convey a forward flow of the pyrolysis product away from the heat-transfer zone and out of the reverse-flow reactor;
(d) at least one fuel conduit in fluidic communication with the combustion zone to convey a reverse flow of a fuel to the combustion zone;
(e) at least one oxidant conduit in fluidic communication with the combustion zone to convey a reverse flow of an oxidant to the combustion zone, wherein
(i) the combustion zone is adapted to combust at least a portion of the fuel with a first portion of the oxidant and convey away from the combustion zone at least (A) a reverse-flow of a first combustion product and (B) a reverse flow of un-combusted oxidant and
(ii) the pyrolysis zone is adapted to oxidize the coke deposits with the un-combusted oxidant flow to produce a second combustion product;
(f) at least one combustion product conduit in fluidic communication with the combustion zone to convey a reverse-flow of the first and second combustion products away from the pyrolysis zone and out of reverse-flow reactor; and
(g) at least one flow controller to (i) establish during a first time interval the forward flows of the gaseous feed and the pyrolysis product and (ii) establish during a second time interval the reverse flows of flow of the fuel, the oxidant, and the combustion product.

24. The reverse flow reactor of claim 23, wherein (i) the reactor vessel has the form of an elongated tube of circular, elliptical, or polygonal cross section, (ii) the elongated tube includes opposed first and second openings, the first opening being in fluidic communication with the feed conduit and the first heat-transfer zone, and the second opening being in fluidic communication with the pyrolysis product conduit and the second heat-transfer zone

25. The reverse flow reactor claim 23, wherein (i) the pyrolysis zone and first heat transfer zone include a first thermal mass for (A) transferring heat to the feed during the first interval and (B) for transferring heat from the combustion product during the second interval, and (ii) the pyrolysis zone and second heat transfer zone include a second thermal mass for (A) transferring heat to the reverse flow of the fuel and/or to the reverse flow the oxidant during the second time interval and (B) for transferring heat away from the pyrolysis product during the first time interval.
